(19) United States
(12) Patent Application Publication
(10) Pub. No.: US 2023/0148574 A1
(43) Pub. Date: May 18, 2023

(54) GENETICALLY MODIFIED NON-HUMAN ANIMAL WITH HUMAN OR CHIMERIC CD38

(71) Applicant: Biocytogen Pharmaceuticals (Beijing) Co., Ltd., Beijing (CN)

(72) Inventors: Yuelei Shen, Beijing (CN); Yang Bai, Beijing (CN); Jiawei Yao, Beijing (CN); Yanan Guo, Beijing (CN); Lei Zhao, Beijing (CN)

(21) Appl. No.: 17/907,590

(22) PCT Filed: Apr. 1, 2021
(86) PCT No.: PCT/CN2021/085053

§ 371 Date: Sep. 28, 2022

(30) Foreign Application Priority Data
Apr. 1, 2020 (CN) 202010251687.1

(51) Int. Cl. A01K 67/027; C07K 14/705; C12N 15/113; G01N 33/50

(52) U.S. Cl. CPC A01K 67/0278; C07K 14/70596; C12N 15/1138; G01N 33/5011; A01K 2217/15; A01K 2227/105; A01K 2267/0306; C12N 2015/8518

(57) ABSTRACT

The present disclosure relates to genetically modified non-human animals that express a human or chimeric (e.g., humanized) CD38, and methods of use thereof.

### CLAIM OF PRIORITY

This application claims the benefit of Chinese Patent Application No. 202010251687.1, filed on Apr. 1, 2020. The entire content of the foregoing application is incorporated herein by reference.

### TECHNICAL FIELD

This disclosure relates to genetically modified animal expressing human or chimeric (e.g., humanized) CD38, and methods of use thereof.

### BACKGROUND

The immune system has developed multiple mechanisms to prevent deleterious activation of immune cells. One such mechanism is the intricate balance between positive and negative costimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

This disclosure is related to an animal model with human CD38 or chimeric CD38. The animal model can express human CD38 or chimeric CD38 (e.g., humanized CD38) protein in its body. It can be used in the studies on the function of CD38 gene, and can be used in the screening and evaluation of anti-human CD38 antibodies. In addition, the animal models prepared by the methods described herein can be used in drug screening, pharmacodynamics studies, treatments for immune-related diseases (e.g., autoimmune disease), and cancer therapy for human CD38 target sites; they can also be used to facilitate the development and design of new drugs, and save time and cost. In summary, this disclosure provides a powerful tool for studying the function of CD38 protein and a platform for screening cancer drugs.

In one aspect, the disclosure is related to a genetically-modified, non-human animal whose genome comprises at least one chromosome comprising a sequence encoding a human or chimeric CD38.

In some embodiments, the sequence encoding the human or chimeric CD38 is operably linked to an endogenous regulatory element at the endogenous CD38 gene locus in the at least one chromosome.

In some embodiments, the sequence encoding a human or chimeric CD38 comprises a sequence encoding an amino acid sequence that is at least 70%, 75%, 80%, 85%, 90%, 95%, 99%, or 100% identical to human CD38 (NP_001766.2 (SEQ ID NO: 4)).

In some embodiments, the sequence encoding a human or chimeric CD38 comprises a sequence encoding an amino acid sequence that is at least 70%, 75%, 80%, 85%, 90%, 95%, 99%, or 100% identical to SEQ ID NO: 13 or 58.

In some embodiments, the sequence encoding a human or chimeric CD38 comprises a sequence that is at least 70%, 75%, 80%, 85%, 90%, 95%, 99%, or 100% identical to amino acids 79-300 of SEQ ID NO: 4. In some embodiments, the sequence encoding a human or chimeric CD38 comprises a sequence that is at least 70%, 75%, 80%, 85%, 90%, 95%, 99%, or 100% identical to amino acids 43-300 of SEQ ID NO: 4.

In some embodiments, the animal is a mammal, e.g., a monkey, a rodent, a rat, or a mouse. In some embodiments, the animal is a mouse.

In some embodiments, the animal does not express endogenous CD38.

In some embodiments, the animal has one or more cells expressing human or chimeric CD38.

In some embodiments, the animal has one or more cells expressing human or chimeric CD38, and human CD31 can bind to the expressed human or chimeric CD38. In some embodiments, the animal has one or more cells expressing human or chimeric CD38, and endogenous CD31 can bind to the expressed human or chimeric CD38.

In one aspect, the disclosure is related to a genetically-modified, non-human animal, in some embodiments, the genome of the animal comprises a replacement of a sequence encoding a region of endogenous CD38 with a sequence encoding all or a portion of the extracellular region of human CD38, thereby generating a chimeric CD38 gene at an endogenous CD38 gene locus.

In some embodiments, the animal is a mouse, and the sequence encoding a region of endogenous CD38 is within exon 1 of the endogenous mouse CD38 gene. In some embodiments, the sequence encoding all or a portion of the extracellular region of human CD38 further comprises Woodchuck Hepatitis Virus (WHP) Posttranscriptional Regulatory Element (WPRE) and/or polyA (polyadenylation) signal sequence.

In some embodiments, the animal is a mouse, and exon 1, exon 2, exon 3, exon 4, exon 5, exon 6, exon 7, and/or exon 8, or a part thereof, of the endogenous mouse CD38 gene is replaced. In some embodiments, a sequence starting within exon 2 and ending within exon 3 of the endogenous mouse CD38 gene is replaced. In some embodiments, the sequence encoding all or a portion of the extracellular region of human CD38 further comprises mouse CD38 3′UTR and/or LoxP STOP sequence.

In some embodiments, the chimeric CD38 gene is operably linked to an endogenous regulatory element at the endogenous CD38 locus, and one or more cells of the animal expresses a chimeric CD38 that is encoded by the chimeric CD38 gene.

In some embodiments, the animal does not express endogenous CD38.

In some embodiments, the animal has one or more cells expressing a chimeric CD38 having a cytoplasmic region, a transmembrane region, and an extracellular region. In some embodiments, the extracellular region comprises a sequence that is at least 50%, 60%, 70%, 80%, 90%, 95%, 99%, or 100% identical to the extracellular region of human CD38. In some embodiments, the extracellular region of the chimeric CD38 has a sequence that has at least 10, 20, 30, 40, 50, 60, 70, 80, 90, 100, 110, 120, 130, 140, 150, 160, 170, 180, 190, 200, 210, 220, 230, 240, or 250 contiguous amino acids that are identical to a contiguous sequence present in the extracellular region of human CD38.

In some embodiments, the animal is heterozygous with respect to the chimeric CD38 gene. In some embodiments, the animal is homozygous with respect to the chimeric CD38 gene.

In one aspect, the disclosure is related to a method for making a genetically-modified, non-human animal, comprising: replacing in at least one cell of the animal, at an endogenous CD38 gene locus, a sequence encoding a region of an endogenous CD38 with a sequence encoding all or a portion of the extracellular region of human CD38.

In some embodiments, the sequence encoding all or a portion of the extracellular region of CD38 comprises exon 1, exon 2, exon 3, exon 4, exon 5, exon 6, exon 7, and/or exon 8, or a part thereof, of a human CD38 gene.

In some embodiments, exon 1, exon 2, exon 3, exon 4, exon 5, exon 6, exon 7, and/or exon 8, or a part thereof, of the endogenous CD38 gene is replaced.

In some embodiments, the sequence encoding all or a portion of the extracellular region of human CD38 encodes amino acids 43-300 of SEQ ID NO: 4. In some embodiments, a sequence within exon 1 of the endogenous CD38 gene is replaced.

In some embodiments, the sequence encoding all or a portion of the extracellular region of human CD38 encodes amino acids 79-300 of SEQ ID NO: 4. In some embodiments, a sequence starting within exon 2 and ending within exon 3 of the endogenous CD38 gene is replaced.

In some embodiments, the animal is a rodent, a rat, or a mouse.

In one aspect, the disclosure is related to a non-human animal comprising at least one cell comprising a nucleotide sequence encoding a chimeric CD38 polypeptide. In some embodiments, the chimeric CD38 polypeptide comprises at least 50 contiguous amino acid residues that are identical to the corresponding contiguous amino acid sequence of a human CD38. In some embodiments, the animal expresses the chimeric CD38 polypeptide.

In some embodiments, the chimeric CD38 polypeptide has at least 50 contiguous amino acid residues that are identical to the corresponding contiguous amino acid sequence of a human CD38 extracellular region. In some embodiments, the chimeric CD38 polypeptide has at least 100 contiguous amino acid residues that are identical to the corresponding contiguous amino acid sequence of a human CD38 extracellular region. In some embodiments, the chimeric CD38 polypeptide has at least 200 contiguous amino acid residues that are identical to the corresponding contiguous amino acid sequence of a human CD38 extracellular region.

In some embodiments, the chimeric CD38 polypeptide comprises a sequence that is at least 80%, 85%, 90%, 95%, or 99% identical to amino acids 43-300 of SEQ ID NO: 4. In some embodiments, the chimeric CD38 polypeptide comprises a sequence that is at least 80%, 85%, 90%, 95%, or 99% identical to amino acids 79-300 of SEQ ID NO: 4.

In some embodiments, the nucleotide sequence is operably linked to an endogenous CD38 regulatory element of the animal.

In some embodiments, the chimeric CD38 polypeptide comprises an endogenous CD38 cytoplasmic region and/or an endogenous CD38 transmembrane region.

In some embodiments, the nucleotide sequence is integrated to an endogenous CD38 gene locus of the animal.

In some embodiments, the chimeric CD38 polypeptide has at least one mouse CD38 activity and/or at least one human CD38 activity.

In one aspect, the disclosure is related to a method of making a genetically-modified mouse cell that expresses a chimeric CD38, the method comprising: replacing at an endogenous mouse CD38 gene locus, a nucleotide sequence encoding a region of mouse CD38 with a nucleotide sequence encoding all or a portion of the extracellular region of human CD38, thereby generating a genetically-modified mouse cell that includes a nucleotide sequence that encodes the chimeric CD38. In some embodiments, the mouse cell expresses the chimeric CD38.

In some embodiments, the chimeric CD38 comprises a cytoplasmic and/or a transmembrane region of mouse CD38; and all or a portion of the extracellular region of human CD38.

In some embodiments, the nucleotide sequence encoding the chimeric CD38 is operably linked to an endogenous CD38 regulatory region, e.g., promoter.

In some embodiments, the animal as described herein further comprises a sequence encoding an additional human or chimeric protein. In some embodiments, the animal expresses the additional human or chimeric protein. In some embodiments, the additional human or chimeric protein is CD31, CD3, B-cell maturation antigen (BCMA), interleukin-15 receptor (IL15R), adenosine A2a receptor (A2aR), programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα), or TNF Receptor Superfamily Member 4 (OX40).

In some embodiments, the animal or mouse as described herein further comprises a sequence encoding an additional human or chimeric protein. In some embodiments, the animal or mouse expresses the additional human or chimeric protein. In some embodiments, the additional human or chimeric protein is CD31, CD3, BCMA, IL15R, A2aR, PD-1, CTLA-4, LAG-3, BTLA, PD-L1, CD27, CD28, CD47, CD137, CD154, TIGIT, TIM-3, GITR, SIRPα, or OX40.

In one aspect, the disclosure is related to a method of determining effectiveness of an anti-CD38 antibody for treating cancer, comprising: administering the anti-CD38 antibody to the animal as described herein, in some embodiments, the animal has a cancer; and determining the inhibitory effects of the anti-CD38 antibody to the cancer.

In some embodiments, the cancer comprises one or more cells that express CD38. In some embodiments, the cancer comprises one or more cancer cells that are injected into the animal.

In some embodiments, determining the inhibitory effects of the anti-CD38 antibody to the cancer involves measuring the tumor volume in the animal.

In some embodiments, the animal has multiple myeloma, acute lymphoblastic leukemia, acute myeloid leukemia, hematological malignancy, solid tumor, and/or non-Hodgkin's lymphoma.

In one aspect, the disclosure is related to a method of determining effectiveness of an anti-CD38 antibody and an additional therapeutic agent for treating cancer, comprising administering the anti-CD38 antibody and the additional therapeutic agent to the animal as described herein, in some embodiments, the animal has a cancer; and determining the inhibitory effects on the cancer.

In some embodiments, the animal further comprises a sequence encoding a human or chimeric programmed cell death protein 1 (PD-1). In some embodiments, the animal further comprises a sequence encoding a human or chimeric programmed death-ligand 1 (PD-L1).

In some embodiments, the additional therapeutic agent is an anti-PD-1 antibody or an anti-PD-L1 antibody.

In some embodiments, the cancer comprises one or more cancer cells that express CD38, PD-L1 or PD-L2.

In some embodiments, the cancer is caused by injection of one or more cancer cells into the animal.

In some embodiments, determining the inhibitory effects of the treatment involves measuring the tumor volume in the animal.

In some embodiments, the animal has melanoma, pancreatic carcinoma, mesothelioma, hematological malignancies (e.g., Non-Hodgkin's lymphoma, lymphoma, chronic lymphocytic leukemia), and/or solid tumors.

In one aspect, the disclosure is related to a method of determining effectiveness of an anti-CD38 antibody for treating an allergic disorder, comprising: administering the anti-CD38 antibody to the animal as described herein, in some embodiments, the animal has the allergic disorder; and determining the inhibitory effects of the anti-CD38 antibody. In some embodiments, the allergic disorder is asthma.

In one aspect, the disclosure is related to a method of determining effectiveness of an anti-CD38 antibody for reducing inflammation, comprising: administering the anti-CD38 antibody to the animal as described herein, in some embodiments, the animal has the inflammation; and determining the inhibitory effects of the anti-CD38 antibody.

In one aspect, the disclosure is related to a method of determining effectiveness of an anti-CD38 antibody for treating autoimmune disorder, comprising: administering the anti-CD38 antibody to the animal as described herein, in some embodiments, the animal has the autoimmune disorder; and determining the inhibitory effects of the anti-CD38 antibody.

In some embodiments, the autoimmune disorder is rheumatoid arthritis, inflammatory bowel disease, systemic lupus erythematosus, or multiple sclerosis.

In one aspect, the disclosure is related to a method of determining toxicity of an anti-CD38 antibody, the method comprising administering the anti-CD38 antibody to the animal as described herein; and determining weight change of the animal. In some embodiments, the method further comprises performing a blood test (e.g., determining red blood cell count).

In one aspect, the disclosure is related to a protein comprising an amino acid sequence. In some embodiments, the amino acid sequence is one of the following: (a) an amino acid sequence set forth in SEQ ID NO: 13 or 58; (b) an amino acid sequence that is at least 90% identical to SEQ ID NO: 13 or 58; (c) an amino acid sequence that is at least 91%, 92%, 93%, 94%, 95%, 96%, 97%, 98%, or 99% identical to SEQ ID NO: 13 or 58; (d) an amino acid sequence that is different from the amino acid sequence set forth in SEQ ID NO: 13 or 58 by no more than 10, 9, 8, 7, 6, 5, 4, 3, 2 or 1 amino acid; and (e) an amino acid sequence that comprises a substitution, a deletion and/or insertion of one, two, three, four, five or more amino acids to the amino acid sequence set forth in SEQ ID NO: 13 or 58.

In one aspect, the disclosure is related to a nucleic acid comprising a nucleotide sequence. In some embodiments, the nucleotide sequence is one of the following: (a) a sequence that encodes the protein as described herein; (b) SEQ ID NO: 8, 9, 12, 50, 51, 54, 55, or 57; (c) a sequence that is at least 90% identical to SEQ ID NO: 8, 9, 12, 50, 51, 54, 55, or 57; and (d) a sequence that is at least 91%, 92%, 93%, 94%, 95%, 96%, 97%, 98%, or 99% identical to SEQ ID NO: 8, 9, 12, 50, 51, 54, 55, or 57.

In one aspect, the disclosure is related to a cell comprising the protein and/or the nucleic acid as described herein. In one aspect, the disclosure is related to an animal comprising the protein and/or the nucleic acid as described herein.

In one aspect, the disclosure is related to a method for making a genetically-modified, non-human animal, comprising: replacing in at least one cell of the animal, at an endogenous locus that encodes an endogenous protein, a sequence encoding a region of the endogenous protein with a sequence encoding all or a portion of the extracellular region of the corresponding protein in human. In some embodiments, the endogenous protein comprises from N-terminus to C-terminus: a cytoplasmic region, a transmembrane region, and an extracellular region.

In some embodiments, the endogenous protein is a type II transmembrane protein. In some embodiments, the endogenous protein is endogenous CD38 and the corresponding protein in human is human CD38.

In one aspect, the disclosure is related to a method for making a genetically-modified, non-human animal, comprising: inserting in at least one cell of the animal, at an endogenous locus that encodes an endogenous protein, a sequence encoding all or a portion of the extracellular region of the corresponding protein in human. In some embodiments, the endogenous protein comprises from N-terminus to C-terminus: a cytoplasmic region, a transmembrane region, and an extracellular region.

In some embodiments, the method further comprises deleting a sequence encoding a region of the endogenous protein.

In some embodiments, the endogenous protein is a type II transmembrane protein. In some embodiments, the endogenous protein is endogenous CD38 and the corresponding protein in human is human CD38.

In one aspect, the disclosure is related to a chimeric CD38 protein, in some embodiments, the chimeric CD38 protein comprises a humanized CD40 extracellular region.

In some embodiments, the chimeric CD38 protein comprises a rodent cytoplasmic region and/or a rodent transmembrane region. In some embodiments, the chimeric CD38 protein comprises a rodent cytoplasmic region and/or a chimeric transmembrane region.

In another aspect, the disclosure also provides a genetically-modified, non-human animal whose genome comprise a disruption in the animal's endogenous CD38 gene, wherein the disruption of the endogenous CD38 gene comprises deletion of exon 1, exon 2, exon 3, exon 4, exon 5, exon 6, exon 7, and/or exon 8, or part thereof of the endogenous CD38 gene (e.g., a region within exon 1; alternatively, a sequence starts within exon 2 and ends within exon 3).

In some embodiments, the disruption of the endogenous CD38 gene comprises deletion of one or more exons or part of exons selected from the group consisting of exon 1, exon 2, exon 3, exon 4, exon 5, exon 6, exon 7, and exon 8 of the endogenous CD38 gene.

In some embodiments, the disruption of the endogenous CD38 gene further comprises deletion of one or more introns or part of introns selected from the group consisting of intron 1, intron 2, intron 3, intron 4, intron 5, intron 6, and intron 7 of the endogenous CD38 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 650, or more nucleotides.

In some embodiments, the disruption of the endogenous CD38 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, and/or exon 8 (e.g., deletion of at least 100 nucleotides of exon 1; alternatively, deletion of at least 250 nucleotides of exon 2 and exon 3).

In some embodiments, the disruption of the endogenous CD38 gene comprises a replacement or an insertion. The replacement or insertion site is after the endogenous regulatory element of the endogenous CD38 gene. In some embodiments, any one or a portion of nucleotide sequence from exon 1 to exon 8 of the endogenous CD38 gene can be replaced or inserted.

In some embodiments, the insertion can destroy the coding frame of the endogenous CD38 gene of the non-human animal, and then an exogenous sequence is inserted. Alternatively, the insertion can cause frameshift mutations in the endogenous CD38 gene and disrupt the endogenous CD38 gene. Alternatively, a termination element is added after the inserted sequence to prevent the expression of non-human animal CD38 protein. In some embodiments, the termination element is a 3′ UTR sequence of endogenous CD38 gene, the LoxP STOP sequence, the WPRE sequence, and/or the polyA sequence as described herein.

In some embodiments, the mice described in the present disclosure can be bred with the mice containing other human or chimeric genes (e.g., CD3, CD28, BCMA, PD-1, PD-L1, IL15R or A2aR), so as to obtain a mouse expressing two or more human or chimeric proteins. The mice can also, e.g., be used for screening antibodies in the case of a combined use of drugs, as well as evaluating the efficacy of the combination therapy.

In another aspect, the disclosure further provides methods of determining toxicity of an agent (e.g., a CD38 antagonist or agonist). The methods involve administering the agent to the animal as described herein; and determining weight change of the animal. In some embodiments, the method further involve performing a blood test (e.g., determining red blood cell count).

The disclosure also relates to non-human mammal generated through the methods as described herein. In some embodiments, the genome thereof contains human gene(s). In some embodiments, the non-human mammal is a rodent. In some embodiments, the non-human mammal is a mouse.

The disclosure further relates to a CD38 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the CD38 gene function, human CD38 antibodies, the drugs or efficacies for human CD38 targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse CD38 gene locus.

FIG. 1B is a schematic diagram showing human CD38 gene locus.

FIG. 2 is a schematic diagram showing humanized CD38 gene locus. chiExon2 includes all or part of human CD38 exons 2-8. chiExon1 and chiExons 3-7 correspond to mouse CD38 exon 1 and exons 4-8, respectively.

FIG. 3 is a schematic diagram showing a CD38 gene targeting strategy.

FIG. 4 shows PCR identification results of constructed targeting vectors. CL-01 to CL-04 are targeting vector numbers. WT is a wild-type control. H₂O is a water control.

FIG. 5 is a schematic diagram showing the FRT recombination process. chiExon2 includes all or part of human CD38 exons 2-8. chiExon1 and chiExons 3-7 correspond to mouse CD38 exon 1 and exons 4-8, respectively.

FIG. 6 is a schematic diagram showing a CD38 gene targeting strategy by CRISPR/Cas gene editing technology.

FIG. 7A shows activity testing results for 5′ end targeting sites of sgRNA1-sgRNA8. PC is positive control. Con is negative control.

FIG. 7B shows activity testing results for 3′ end targeting sites of sgRNA9-sgRNA15. PC is positive control. Con is negative control.

FIG. 8A shows PCR identification results of F0 generation mice by primers L-GT-F and L-GT-R. WT is a wild-type control. H₂O is a water control.

FIG. 8B shows PCR identification results of F0 generation mice by primers R-GT-F and R-GT-R. WT is a wild-type control. H₂O is a water control.

FIG. 9A shows PCR identification results of F1 generation mice by primers L-GT-F and L-GT-R. WT is a wild-type control. H₂O is a water control. M is a marker. F1-01 to F1-12 are mouse numbers.

FIG. 9B shows PCR identification results of F1 generation mice by primers R-GT-F and R-GT-R. WT is a wild-type control. H₂O is a water control. M is a marker. F1-01 to F1-12 are mouse numbers.

FIG. 10 shows Southern Blot analysis result of F1 generation mice by P1 or P2 probe. F1-01 to F1-09 are mouse numbers. WT is a wild-type control.

FIG. 11A is a flow cytometry result of spleen cells collected from a wild-type C57BL/6 mouse. The cells were labeled with anti-mouse CD38 antibody mCD38-BV421-A and anti-mouse CD19 antibody mCD19-FITC-A.

FIG. 11B is a flow cytometry result of spleen cells collected from a CD38 gene humanized heterozygous mouse (B-hCD38 (H/+)). The cells were labeled with anti-mouse CD38 antibody mCD38-BV421-A and anti-mouse CD19 antibody mCD19-FITC-A.

FIG. 11C is a flow cytometry result of spleen cells collected from a wild-type C57BL/6 mouse. The cells were labeled with anti-human CD38 antibody hCD38-APC-A and anti-mouse CD19 antibody mCD19-FITC-A.

FIG. 11D is a flow cytometry result of spleen cells collected from a CD38 gene humanized heterozygous mouse (B-hCD38 (H/+)). The cells were labeled with anti-human CD38 antibody hCD38-APC-A and anti-mouse CD19 antibody mCD19-FITC-A.

FIG. 12 shows PCR identification results of CD38 gene knockout mice. 01 to 11 are mouse numbers. M is a marker. WT is a wild-type control. H₂O is a water control.

FIG. 13 is a schematic diagram showing humanized CD38 gene locus.

FIG. 14 is a schematic diagram showing a CD38 gene targeting strategy.

FIG. 15A is a flow cytometry result of spleen cells collected from a wild-type C57BL/6 mouse. The cells were labeled with anti-mouse CD45 antibody Brilliant Violet 510™ anti-mouse CD45 Antibody, anti-mouse CD19 antibody mCD19-FITC-A, and anti-mouse CD38 antibody mCD38-BV421-A.

FIG. 15B is a flow cytometry result of spleen cells collected from a CD38 gene humanized homozygous mouse (B-hCD38 (H/H)). The cells were labeled with anti-mouse CD45 antibody Brilliant Violet 510™ anti-mouse CD45 Antibody, anti-mouse CD19 antibody mCD19-FITC-A, and anti-mouse CD38 antibody mCD38-BV421-A.

FIG. 15C is a flow cytometry result of spleen cells collected from a wild-type C57BL/6 mouse. The cells were labeled with anti-human CD45 antibody Brilliant Violet 510™ anti-mouse CD45 Antibody, anti-mouse CD19 antibody mCD19-FITC-A, and anti-human CD38 antibody hCD38-PE-A.

FIG. 15D is a flow cytometry result of spleen cells collected from a CD38 gene humanized homozygous mouse (B-hCD38 (H/H)). The cells were labeled with anti-human CD45 antibody Brilliant Violet 510™ anti-mouse CD45 Antibody, anti-mouse CD19 antibody mCD19-FITC-A, and anti-human CD38 antibody hCD38-PE-A.

FIG. 16A is a flow cytometry result of blood cells collected from a wild-type C57BL/6 mouse. The cells were labeled with anti-mouse CD45 antibody Brilliant Violet 510™ anti-mouse CD45 Antibody, anti-mouse CD19 antibody mCD19-FITC-A, and anti-mouse CD38 antibody mCD38-BV421-A.

FIG. 16B is a flow cytometry result of blood cells collected from a CD38 gene humanized homozygous mouse (B-hCD38 (H/H)). The cells were labeled with anti-mouse CD45 antibody Brilliant Violet 510™ anti-mouse CD45 Antibody, anti-mouse CD19 antibody mCD19-FITC-A, and anti-mouse CD38 antibody mCD38-BV421-A.

FIG. 16C is a flow cytometry result of blood cells collected from a wild-type C57BL/6 mouse. The cells were labeled with anti-human CD45 antibody Brilliant Violet 510™ anti-mouse CD45 Antibody, anti-mouse CD19 antibody mCD19-FITC-A, and anti-human CD38 antibody hCD38-PE-A.

FIG. 16D is a flow cytometry result of blood cells collected from a CD38 gene humanized homozygous mouse (B-hCD38 (H/H)). The cells were labeled with anti-human CD45 antibody Brilliant Violet 510™ anti-mouse CD45 Antibody, anti-mouse CD19 antibody mCD19-FITC-A, and anti-human CD38 antibody hCD38-PE-A.

FIG. 17 shows the alignment between mouse CD38 amino acid sequence (NP_031672.2; SEQ ID NO: 2) and human CD38 amino acid sequence (NP_001766.2; SEQ ID NO: 4).

FIG. 18 shows the alignment between rat CD38 amino acid sequence (NP_037259.1; SEQ ID NO: 59) and human CD38 amino acid sequence (NP_001766.2; SEQ ID NO: 4).

### DETAILED DESCRIPTION

This disclosure relates to transgenic non-human animal with human or chimeric (e.g., humanized) CD38, and methods of use thereof.

CD38 (cluster of differentiation 38), also known as cyclic ADP ribose hydrolase is a glycoprotein found on the surface of many immune cells (white blood cells), including CD4+, CD8+, B lymphocytes and natural killer cells. CD38 also functions in cell adhesion, signal transduction and calcium signaling. CD38 can function either as a receptor or as an enzyme. As a receptor, CD38 can attach to CD31 on the surface of T cells, thereby activating those cells to produce a variety of cytokines.

CD38 is a multifunctional enzyme that catalyzes the synthesis of ADP ribose (ADPR) (97%) and cyclic ADP-ribose (cADPR) (3%) from NAD+. CD38 can be a major regulator of NAD+ levels because 100 molecules of NAD+ is required to generate one molecule of cADPR. CD38 also hydrolyzes cADPR to ADPR. When nicotinic acid is present under acidic conditions, CD38 can hydrolyze nicotinamide adenine dinucleotide phosphate (NADP+) to NAADP. These reaction products are essential for the regulation of intracellular Ca²⁺. CD38 occurs not only as an ectoezyme on cell outer surfaces, but also occurs on the inner surface of cell membranes, facing the cytosol performing the same enzymatic functions. CD38 is believed to control or influence neurotransmitter release in the brain by producing cADPR. CD38 within the brain enables release of the affiliative neuropeptide oxytocin.

The loss of CD38 function is associated with impaired immune responses, metabolic disturbances, and behavioral modifications including social amnesia possibly related to autism. CD31 on endothelial cells binds to the CD38 receptor on natural killer cells for those cells to attach to the endothelium. CD38 on leukocytes attaching to CD16 on endothelial cells allows for leukocyte binding to blood vessel walls, and the passage of leukocytes through blood vessel walls. The cytokine interferon gamma and the Gram negative bacterial cell wall component lipopolysaccharide induce CD38 expression on macrophages. Interferon gamma strongly induces CD38 expression on monocytes. The cytokine tumor necrosis factor strongly induces CD38 on airway smooth muscle cells inducing cADPR-mediated Ca′, thereby increasing dysfunctional contractility resulting in asthma.

The CD38 protein is a marker of cell activation. It has been connected to HIV infection, leukemias, myelomas, solid tumors, type II diabetes mellitus and bone metabolism, as well as some genetically determined conditions. CD38 increases airway contractility hyperresponsiveness, is increased in the lungs of asthmatic patients, and amplifies the inflammatory response of airway smooth muscle of those patients. Increased expression of CD38 is an unfavourable diagnostic marker in chronic lymphocytic leukemia and is associated with increased disease progression. Thus, CD38 antibodies can be potentially used as cancer therapies, allergic or autoimmune disorders.

Experimental animal models are an indispensable research tool for studying the effects of these antibodies (e.g., CD38 antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullis et al U.S. Pat. No. 4,683,195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N. Y., 1986); each of which is incorporated herein by reference in its entirety.

### CD38

Human CD38 Ag is a type II 45-kDa transmembrane glycoprotein, prevalently expressed by immature and activated T and B lymphocytes, plasma cells, monocytes, peripheral blood NK cells and, at a lower epitope density, other cells and tissues.

CD38 is a multifunctional transmembrane protein that is widely expressed in immune cells. In lymphocytes, monocytes, macrophages, dendritic cells, granulocytes, and natural killer (NK) cells, the expression levels of CD38 on the cell surface depend on the stage of maturation and/or activation of the cell. Since its discovery almost four decades ago, accumulated evidence indicates that CD38 plays important roles in various cell types in both physiological and pathological contexts. Early studies suggested that human CD38 can establish lateral associations with various membrane proteins/complexes, such as CD16 (in NK cells), the T cell receptor (TCR)/CD3 complex and CD4 (in T cells), membrane immunoglobulin (Ig) and the B cell co-receptor complex (CD19/CD81) (in B lymphocytes), and class II MHC (in monocytes). On the basis of such interactions, CD38 was proposed to potentially contribute to cell signaling from these complexes. In agreement with this notion, and despite the fact that CD38 contains a short cytoplasmic domain without signaling motifs, CD38 relocalized at the immunologic synapse in T cells upon TCR engagement, contributing to modulation of antigen-mediated T-cell responses. In the same line, CD38 crosslinking decreased the threshold for B cell activation via the B-cell receptor (BCR), suggesting its participation in BCR signaling. Human CD38 was also shown to bind a specific nonsubstrate ligand, CD31/PECAM-1, a member of the Ig superfamily that is highly expressed on the surface of different cell types, including endothelial cells. Interference with CD38-CD31 interaction inhibited lymphocyte adhesion to endothelial cells.

In addition to receptor or co-receptor functions, CD38 also plays multiple roles derived from intrinsic enzymatic activities. At neutral pH, CD38 converts nicotinamide adenine dinucleotide (NAD) into ADP ribose (ADPR), cyclic ADPR (cADPR) and nicotinamide, whereas at acidic pH, CD38 uses NAD phosphate (NADP) to generate nicotinic acid adenine dinucleotide phosphate. The enzymatic products of these reactions are calcium-mobilizing second messengers with relevant signaling consequences in diverse cellular contexts. Because the generation of ADPR and cADPR requires large consumption of NAD, CD38 is considered the major NAD glycohydrolase (NADase) in mammalian tissues. In addition, CD38 can also catabolize the extracellular NAD+ precursors nicotinamide mononucleotide and nicotinamide riboside before they are transported into the cell for NAD+ biosynthesis.

The consequences of CD38 expression depend also on the ultrastructural configuration of the molecule and its location within the cell. CD38 has been shown to exist as either monomeric, dimeric or even multimeric type II forms, displaying the catalytic site outside of the cell, and as a type III form with the catalytic site facing the cytoplasm. Moreover, an intracellular pool of CD38 has been shown to be associated to mitochondrial and nuclear membranes. In these configurations, CD38 could have access to both extracellular and intracellular NAD+. In addition, CD38 also exists as a soluble form, which is detectable in biological fluids.

A detailed description of CD38 and its function can be found, e.g., in Glaria, Estibaliz, and Annabel F. Valledor. “Roles of CD38 in the Immune Response to Infection.” Cells 9.1 (2020): 228; Lee, Hon Cheung. “Structure and enzymatic functions of human CD38.” Molecular medicine 12.11 (2006): 317-323; Hogan, Kelly A. et al., “The multi-faceted ecto-enzyme CD38: roles in immunomodulation, cancer, aging, and metabolic diseases.” Frontiers in Immunology 10 (2019): 1187; each of which is incorporated by reference in its entirety.

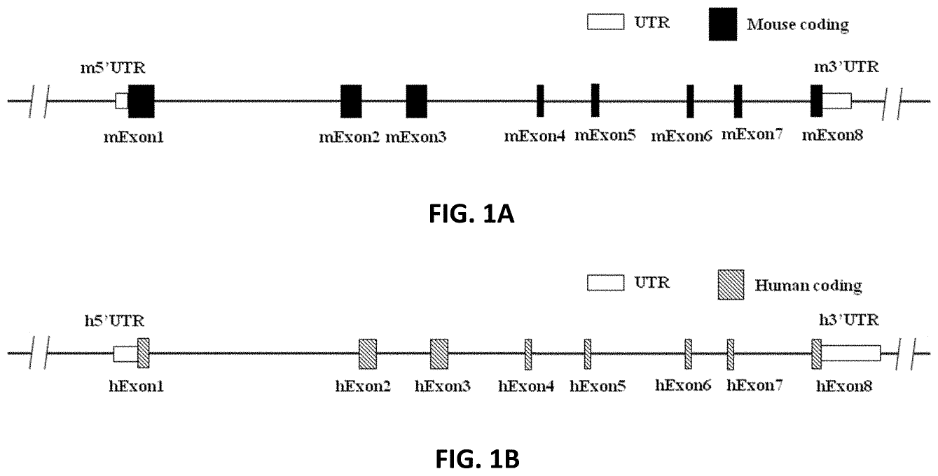

In human genomes, CD38 gene (Gene ID: 952) locus has eight exons, exon 1, exon 2, exon 3, exon 4, exon 5, exon 6, exon 7, and exon 8 (FIG. 1B). The CD38 protein also has, from N-terminus to C-terminus, a cytoplasmic region, a transmembrane region, and an extracellular region. The nucleotide sequence for human CD38 mRNA is NM 001775.4 (SEQ ID NO: 3), and the amino acid sequence for human CD38 is NP_001766.2 (SEQ ID NO: 4). The location for each exon and each region in human CD38 nucleotide sequence and amino acid sequence is listed below:

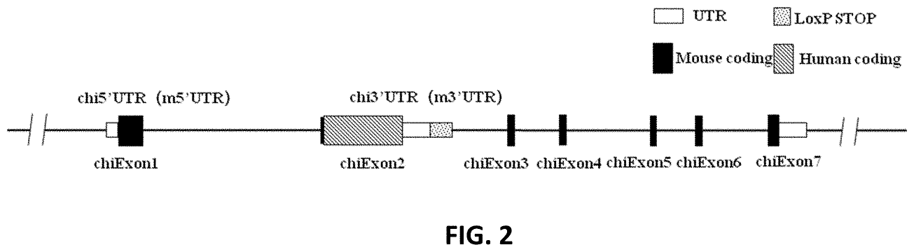

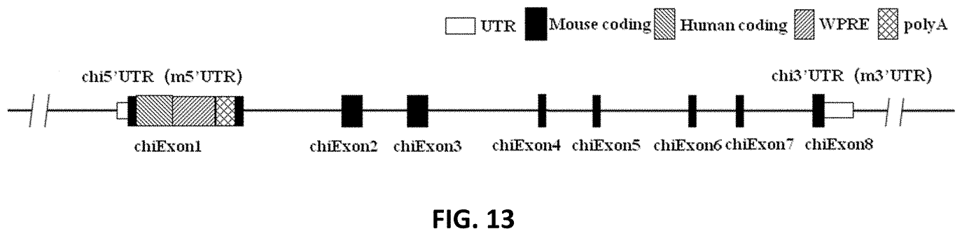

TABLE 1

| | NM_001775.4 | NP_001766.2 |
|---|---|---|
| Human CD38 | 5620bp | 300aa |
| (approximate location) | (SEQ ID NO: 3) | (SEQ ID NO: 4) |
| Exon 1 | 1-320 | 1-78 |
| Exon 2 | 321-450 | 79-121 |
| Exon 3 | 451-586 | 122-166 |
| Exon 4 | 587-672 | 167-195 |
| Exon 5 | 673-746 | 196-220 |
| Exon 6 | 747-839 | 221-251 |
| Exon 7 | 840-926 | 252-280 |
| Exon 8 | 927-5620 | 281-300 |
| Cytoplasmic | 1-150 | 1-21 |
| Transmembrane region | 151-213 | 22-42 |
| Extracellular | 214-5620 | 43-300 |
| Donor region in FIG. 2 | 322-990 | 79-300 |
| Donor region in FIG. 13 | 214-990 | 43-300 |

In mice, CD38 gene locus has eight exons, exon 1, exon 2, exon 3, exon 4, exon 5, exon 6, exon 7, and exon 8 (FIG. 1A). The mouse CD38 protein also has a cytoplasmic region, a transmembrane region, and an extracellular region. The nucleotide sequence for mouse CD38 mRNA is NM_007646.5 (SEQ ID NO: 1), the amino acid sequence for mouse CD38 is NP_031672.2 (SEQ ID NO: 2). The location for each exon and each region in the mouse CD38 nucleotide sequence and amino acid sequence is listed below:

TABLE 2

| | NM_007646.5 | NP_031672.2 |
|---|---|---|
| Mouse CD38 | 3013bp | 304aa |
| (approximate location) | (SEQ ID NO: 1) | (SEQ ID NO: 2) |
| Exon 1 | 1-312 | 1-82 |
| Exon 2 | 313-442 | 83-125 |
| Exon 3 | 443-578 | 126-170 |
| Exon 4 | 579-664 | 171-199 |
| Exon 5 | 665-738 | 191-224 |
| Exon 6 | 739-831 | 225-255 |
| Exon 7 | 832-918 | 256-284 |
| Exon 8 | 919-3013 | 285-304 |
| Cytoplasmic | 1-130 | 1-21 |
| Transmembrane region | 131-199 | 22-44 |
| Extracellular | 200-3013 | 45-304 |
| Replaced region in FIG. 2 | 314-577 | 83-170 |
| Replaced region in FIG. 13 | 194-310 | 43-81 |

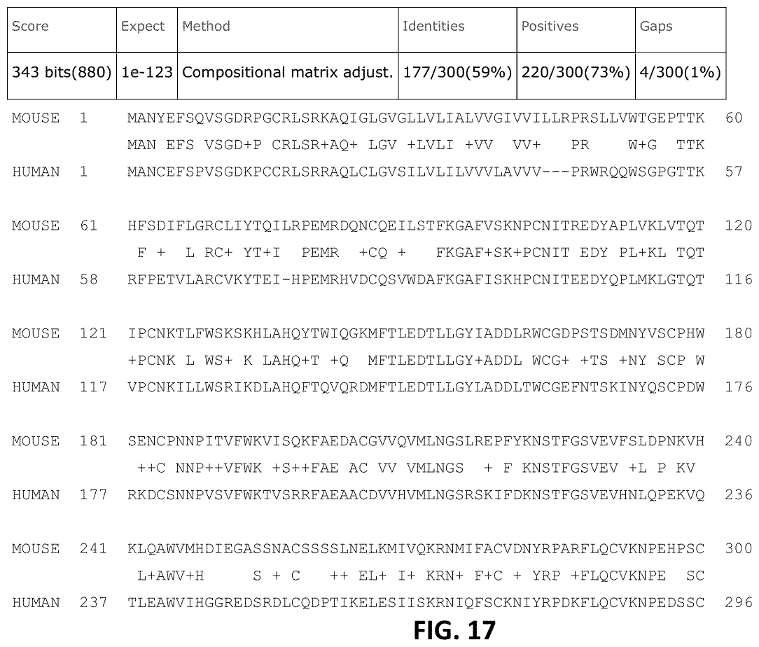

The mouse CD38 gene (Gene ID: 12494) is located in Chromosome 5 of the mouse genome, which is located from 43868809 to 43912374 of NC_000071.6 (GRCm38.p6 (GCF_000001635.26)). The 5′-UTR is from 43,868,553 to 43,868,875, exon 1 is from 43,868,553 to 43,869,120, the first intron is from 43,869,121 to 43,900,332, exon 2 is from 43,900,333 to 43,900,462, the second intron is from 43,900,463 to 43,901,420, exon 3 is from to 43,901,556, the third intron is from 43,901,557 to 43,903,594, exon 4 is from to 43,903,680, the fourth intron is from 43,903,681 to 43,906,163, exon 5 is from 43,906,164 to 43,906,237, the fifth intron is from 43,906,238 to 43,907,512, exon 6 is from 43,907,513 to 43,907,605, the sixth intron is from 43,907,606 to 43,907,922, exon 7 is from 43,907,923 to 43,908,009, the seventh intron is from 43,908,010 to 43,910,279, exon 8 is from 43,910,280 to 43,912,375, the 3′-UTR is from 43,910,404 to 43,912,375, based on transcript NM_007646.5 (SEQ ID NO: 1). All relevant information for mouse CD38 locus can be found in the NCBI website with Gene ID: 12494, which is incorporated by reference herein in its entirety. FIG. 17 shows the alignment between mouse CD38 amino acid sequence (NP_031672.2; SEQ ID NO: 2) and human CD38 amino acid sequence (NP_001766.2; SEQ ID NO: 4). Thus, the corresponding amino acid residue or region between mouse and human CD38 can be found in FIG. 17.

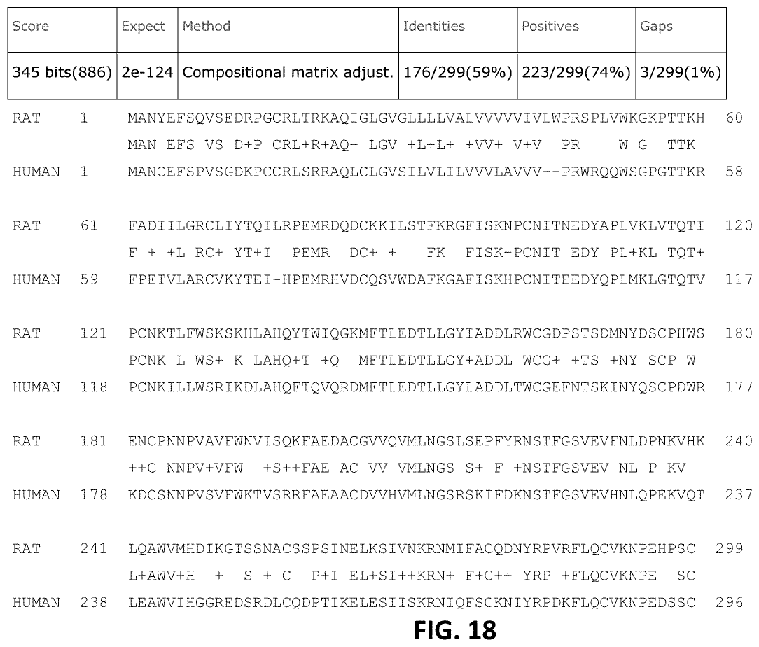

CD38 genes, proteins, and locus of the other species are also known in the art. For example, the gene ID for CD38 in Rattus norvegicus is 25668, the gene ID for CD38 in Macaca mulatta (Rhesus monkey) is 714399, the gene ID for CD38 in Canis lupus familiaris (dog) is 403756, and the gene ID for CD38 in Sus scrofa (pig) is 100511702. The relevant information for these genes (e.g., intron sequences, exon sequences, amino acid residues of these proteins) can be found, e.g., in NCBI database, which is incorporated by reference herein in its entirety. FIG. 18 shows the alignment between rodent CD38 amino acid sequence (NP_037259.1; SEQ ID NO: 59) and human CD38 amino acid sequence (NP_001766.2; SEQ ID NO: 4). Thus, the corresponding amino acid residue or region between rodent and human CD38 can be found in FIG. 18.

The present disclosure provides human or chimeric (e.g., humanized) CD38 nucleotide sequence and/or amino acid sequences. In some embodiments, the entire sequence of mouse exon 1, exon 2, exon 3, exon 4, exon 5, exon 6, exon 7, exon 8, cytoplasmic region, transmembrane region, and/or extracellular region are replaced by the corresponding human sequence. In some embodiments, a “region” or “portion” of mouse exon 1, exon 2, exon 3, exon 4, exon 5, exon 6, exon 7, exon 8, cytoplasmic region, transmembrane region, and/or extracellular region are replaced by the corresponding human sequence. In some embodiments, a “region” or “portion” of mouse exon 1, exon 2, exon 3, exon 4, exon 5, exon 6, exon 7, exon 8, cytoplasmic region, transmembrane region, and/or extracellular region are replaced by a sequence encoding a “region” or “portion” of the extracellular region of human CD38. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides (contiguous or non-contiguous), or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues (contiguous or non-contiguous). In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, cytoplasmic region, transmembrane region, or extracellular region. In some embodiments, a region, a portion, or the entire sequence of mouse exon 1, exon 2, exon 3, exon 4, exon 5, exon 6, exon 7, and/or exon 8 (e.g., a region within exon 1; alternatively, exon 2 and exon 3) are replaced by a sequence comprising the human exon 1, exon 2, exon 3, exon 4, exon 5, exon 6, exon 7, and/or exon 8 (e.g., a region within exon 1, exon 2, exon 3, exon 4, exon 5, exon 6, exon 7, and exon 8; alternatively, exon 2, exon 3, exon 4, exon 5, exon 6, exon 7, and exon 8) sequence.

In some embodiments, the present disclosure also provides a chimeric (e.g., humanized) CD38 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CD38 mRNA sequence (e.g., SEQ ID NO: 1), mouse CD38 amino acid sequence (e.g., SEQ ID NO: 2), or a portion thereof (e.g., all or a portion of exon 1); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CD38 mRNA sequence (e.g., SEQ ID NO: 3), human CD38 amino acid sequence (e.g., SEQ ID NO: 4), or a portion thereof (e.g., a region within exon 1, exon 2, exon 3, exon 4, exon 5, exon 6, exon 7, and exon 8; alternatively, exon 2, exon 3, exon 4, exon 5, exon 6, exon 7, and exon 8).

In some embodiments, the sequence encoding amino acids 43-81 of mouse CD38 (SEQ ID NO: 2) is replaced or inactivated. In some embodiments, the sequence is replaced by a sequence comprising all or a portion of the extracellular region of human CD38 (e.g., amino acids 43-300 of human CD38 (SEQ ID NO: 4)).

In some embodiments, the sequence encoding amino acids 83-170 of mouse CD38 (SEQ ID NO: 2) is replaced or inactivated. In some embodiments, the sequence is replaced by a sequence comprising all or a portion of the extracellular region of human CD38 (e.g., amino acids 79-300 of human CD38 (SEQ ID NO: 4)). In some embodiments, the sequence is replaced by a sequence comprising 1, 2, 3, 4, 5, 6, 7, 8, 9, 10, 11, 12, 13, 14, 15, 17, 18, 19, 20, 21, 22, 23, 24, 25, 26, 27, 28, 29, 30, 31, 32, 33, 34, 35, or 36 residues (contiguous or non-contiguous) of amino acids 43-78 of SEQ ID NO: 4.

In some embodiments, the nucleic acids as described herein are operably linked to a promotor or regulatory element, e.g., an endogenous mouse CD38 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse CD38 nucleotide sequence (e.g., exon 1, exon 2, exon 3, exon 4, exon 5, exon 6, exon 7, exon 8, and/or NM_007646.5 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse CD38 nucleotide sequence (e.g., exon 1, exon 2, exon 3, exon 4, exon 5, exon 6, exon 7, exon 8, and/or NM_007646.5 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human CD38 nucleotide sequence (e.g., exon 1, exon 2, exon 3, exon 4, exon 5, exon 6, exon 7, exon 8, and/or NM_001775.4 (SEQ ID NO: 3)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human CD38 nucleotide sequence (e.g., exon 1, exon 2, exon 3, exon 4, exon 5, exon 6, exon 7, exon 8, and/or NM_001775.4 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse CD38 amino acid sequence (e.g., amino acid sequence encoded by exon 1, exon 2, exon 3, exon 4, exon 5, exon 6, exon 7, exon 8, and/or NP_031672.2 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse CD38 amino acid sequence (e.g., amino acid sequence encoded by exon 1, exon 2, exon 3, exon 4, exon 5, exon 6, exon 7, exon 8, and/or NP_031672.2 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human CD38 amino acid sequence (e.g., amino acid sequence encoded by exon 1, exon 2, exon 3, exon 4, exon 5, exon 6, exon 7, exon 8, and/or NP_001766.2 (SEQ ID NO: 4)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human CD38 amino acid sequence (e.g., amino acid sequence encoded by exon 1, exon 2, exon 3, exon 4, exon 5, exon 6, exon 7, exon 8, and/or NP_001766.2 (SEQ ID NO: 4)).

In some embodiments, the chimeric CD38 gene as described herein comprises a sequence comprising a portion of intron 3, and the entirety of exons 4-8 of the endogenous CD38 gene of the non-human animal; preferably, the exons 4-8 of the endogenous CD38 gene of the non-human animal has a homology of at least 50%, 60%, 70%, 80%, 90%, or 95% with the corresponding exons 4-8 of NC_000071.6; more preferably, the exons 4-8 of the endogenous CD38 gene of the non-human animal is identical to exons 4-8 of SEQ ID NO: 1.

In some embodiments, the chimeric CD38 gene as described herein comprises a sequence comprising the entirety or part of exons 2-8 of the endogenous CD38 gene of the non-human animal; preferably, the exons 2-8 of the endogenous CD38 gene of the non-human animal has a homology of at least 50%, 60%, 70%, 80%, 90%, or 95% with the corresponding exons 2-8 of NC_000071.6; more preferably, the exons 2-8 of the endogenous CD38 gene of the non-human animal is identical to exons 2-8 of SEQ ID NO: 1.

In some embodiments, the chimeric CD38 gene as described herein comprises a sequence comprising: the entirety of exon 1, intron 1, the first nucleotide of exon 2 of the endogenous CD38 gene; the cDNA sequence derived from exons 2-8 of human CD38 gene; the 3′UTR of endogenous CD38 gene; and a LoxP STOP sequence. Preferably, the LoxP STOP sequence is followed by the entirety of exons 4-8 of the endogenous CD38 gene. More preferably, the chimeric CD38 gene further comprises a portion of intron 3 of the endogenous CD38 gene.

In some embodiments, the chimeric CD38 gene as described herein comprises a sequence comprising: a nucleotide sequence encoding the cytoplasmic and transmembrane region of endogenous CD38; the cDNA sequence derived from exons 1-8 of human CD38 gene encoding the extracellular region of human CD38; and the WPRE-polyA sequence. Preferably, the WPRE-polyA sequence is followed by the nucleotide sequence encoding the last amino acid of exon 1, intron 1, and the entirety of exons 2-8 of the endogenous CD38 gene.

The present disclosure also provides a humanized CD38 mouse amino acid sequence, wherein the amino acid sequence is selected from the group consisting of:

a) an amino acid sequence comprising all or a portion of amino acids 43-300 or 79-300 of SEQ ID NO: 4;

b) an amino acid sequence having a homology of at least 70%, 75%, 80%, 85%, 90%, 91%, 92%, 93%, 94%, 95%, 96%, 97%, 98%, or 99% identical to amino acids 43-300 or 79-300 of SEQ ID NO: 4;

(c) an amino acid sequence that is different from the amino acid sequence shown in amino acids 43-300 or 79-300 of SEQ ID NO: 4 by no more than 10, 9, 8, 7, 6, 5, 4, 3, 2 or no more than 1 amino acid; or

(d) an amino acid sequence that comprises a substitution, a deletion and/or insertion of one or more amino acids to amino acids 43-300 or 79-300 of SEQ ID NO: 4.

The present disclosure also provides a humanized CD38 mouse amino acid sequence, wherein the amino acid sequence is selected from the group consisting of:

a) an amino acid sequence shown in SEQ ID NO: 13 or 58;

b) an amino acid sequence having a homology of at least 90% with or at least 90% identical to the amino acid sequence shown in SEQ ID NO: 13 or 58;

c) an amino acid sequence encoded by a nucleic acid sequence, wherein the nucleic acid sequence is able to hybridize to a nucleotide sequence encoding the amino acid shown in SEQ ID NO: 13 or 58 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 13 or 58;

e) an amino acid sequence that is different from the amino acid sequence shown in SEQ ID NO: 13 or 58 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 13 or 58.

The present disclosure also relates to a CD38 nucleic acid (e.g., DNA or RNA) sequence, wherein the nucleic acid sequence can be selected from the group consisting of:

a) a nucleotide sequence comprising 322-990 of SEQ ID NO: 3;

b) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence of 322-990 of SEQ ID NO:3; or

c) a nucleic acid sequence that comprises at least or about 1, 2, 3, 4, 5, 6, 7, 8, 9, 10, 20, 30, 40, 50, 60, 70, 80, 90, 100, or 110 nucleotides (contiguous or non-contiguous) of 214-321 of SEQ ID NO: 3.

The present disclosure also relates to a CD38 nucleic acid (e.g., DNA or RNA) sequence, wherein the nucleic acid sequence can be selected from the group consisting of:

a) a nucleic acid sequence as shown in SEQ ID NO: 8, 9, 12, 50, 51, 54, 55, or 57, or a nucleic acid sequence encoding a homologous CD38 amino acid sequence of a humanized mouse;

b) a nucleic acid sequence that is shown in SEQ ID NO: 8, 9, 12, 50, 51, 54, 55, or 57;

c) a nucleic acid sequence that is able to hybridize to the nucleotide sequence as shown in SEQ ID NO: 8, 9, 12, 50, 51, 54, 55, or 57 under a low stringency condition or a strict stringency condition;

d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 8, 9, 12, 50, 51, 54, 55, or 57;

e) a nucleic acid sequence that encodes an amino acid sequence, wherein the amino acid sequence has a homology of at least 90% with or at least 90% identical to the amino acid sequence shown in SEQ ID NO: 13 or 58;

f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 13 or 58;

g) a nucleic acid sequence that encodes an amino acid sequence, wherein the amino acid sequence is different from the amino acid sequence shown in SEQ ID NO: 13 or 58 by no more than 10, 9, 8, 7, 6, 5, 4, 3, 2 or no more than 1 amino acid; and/or

h) a nucleic acid sequence that encodes an amino acid sequence, wherein the amino acid sequence comprises a substitution, a deletion and/or insertion of one or more amino acids to the amino acid sequence shown in SEQ ID NO: 13 or 58.

The present disclosure further relates to a CD38 genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 8, 9, 12, 50, 51, 54, 55, or 57.

The disclosure also provides an amino acid sequence that has a homology of at least 90% with, or at least 90% identical to the sequence shown in SEQ ID NO: 27, and has protein activity. In some embodiments, the homology with the sequence shown in SEQ ID NO: 13 or 58 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 13 or 58 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 8, 9, 12, 50, 51, 54, 55, or 57, and encodes a polypeptide that has protein activity. In some embodiments, the homology with the sequence shown in SEQ ID NO: 8, 9, 12, 50, 51, 54, 55, or 57 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 8, 9, 12, 50, 51, 54, 55, or 57 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) CD38 from an endogenous non-human CD38 locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous CD38 locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be e.g., a human exon or a chimeric exon.

In some embodiments, the chimeric gene or the chimeric nucleic acid is a humanized CD38 gene or a humanized CD38 nucleic acid. In some embodiments, at least one or more portions of the gene or the nucleic acid is from the human CD38 gene, at least one or more portions of the gene or the nucleic acid is from a non-human CD38 gene. In some embodiments, the gene or the nucleic acid comprises a sequence that encodes a CD38 protein. The encoded CD38 protein is functional or has at least one activity of the human CD38 protein or the non-human CD38 protein, e.g., binding with human or non-human CD38 ligand (e.g., CD31), activating T cells to produce cytokines, hydrolyzing cADPR to ADPR, hydrolyzing NADP+ to NAADP, regulating intracellular Ca²⁺, controlling neurotransmitter release, catalyzing the synthesis of ADP ribose (ADPR) and cyclic ADP-ribose (cADPR) from NAD+, regulating NAD+ levels, regulating lymphocyte adhesion, proliferation and cytokine production, and/or upregulating the immune response.

In some embodiments, the chimeric protein or the chimeric polypeptide is a humanized CD38 protein or a humanized CD38 polypeptide. In some embodiments, at least one or more portions of the amino acid sequence of the protein or the polypeptide is from a human CD38 protein, and at least one or more portions of the amino acid sequence of the protein or the polypeptide is from a non-human CD38 protein. The humanized CD38 protein or the humanized CD38 polypeptide is functional or has at least one activity of the human CD38 protein or the non-human CD38 protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from BALB/c, A, A/He, A/J, A/WySN, AKR, AKR/A, AKR/J, AKR/N, TA1, TA2, RF, SWR, C3H, C57BR, SJL, C57L, DBA/2, KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, C57BL/Ola, C57BL, C58, CBA/Br, CBA/Ca, CBA/J, CBA/st, and CBA/H. In some embodiments, the mouse is a 129 strain selected from the group consisting of a strain that is 129P1, 129P2, 129P3, 129X1, 129S1 (e.g., 12951/SV, 12951/SvIm), 129S2, 129S4, 129S5, 12959/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized CD38 animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Ry knockout mice, NOD/SCID/γcnull mice (Ito, M. et al., NOD/SCID/γcnull mouse: an excellent recipient mouse model for engraftment of human cells, Blood 100(9): 3175-3182, 2002), NOD-Prkdc^{scid} IL-2rg^{null} mice, nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human CD38 locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Ry knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature CD38 coding sequence with human mature CD38 coding sequence.

Genetically modified non-human animals that comprise a modification of an endogenous non-human CD38 locus. In some embodiments, the modification can comprise a human nucleic acid sequence encoding at least a portion of a mature CD38 protein (e.g., at least 10%, 20%, 30%, 40%, 50%, 60%, 70%, 80%, 90%, 95%, 96%, 97%, 98%, or 99% identical to the mature CD38 protein sequence). Although genetically modified cells are also provided that can comprise the modifications described herein (e.g., ES cells, somatic cells), in many embodiments, the genetically modified non-human animals comprise the modification of the endogenous CD38 locus in the germline of the animal.

Genetically modified animals can express a human CD38 and/or a chimeric (e.g., humanized) CD38 from endogenous mouse loci, wherein the endogenous mouse CD38 gene has been replaced with a human CD38 gene and/or a nucleotide sequence that encodes a region of human CD38 sequence or an amino acid sequence that is at least 10%, 20%, 30%, 40%, 50%, 60%, 70&, 80%, 90%, 95%, 96%, 97%, 98%, or 99% identical to the human CD38 sequence. In various embodiments, an endogenous non-human CD38 locus is modified in whole or in part to comprise human nucleic acid sequence encoding at least one protein-coding sequence of a mature CD38 protein.

In some embodiments, the genetically modified mice express the human CD38 and/or chimeric CD38 (e.g., humanized CD38) from endogenous loci that are under control of mouse promoters and/or mouse regulatory elements. The replacement(s) at the endogenous mouse loci provide non-human animals that express human CD38 or chimeric CD38 (e.g., humanized CD38) in appropriate cell types and in a manner that does not result in the potential pathologies observed in some other transgenic mice known in the art. The human CD38 or the chimeric CD38 (e.g., humanized CD38) expressed in animal can maintain one or more functions of the wild-type mouse or human CD38 in the animal. For example, human or non-human CD38 ligands (e.g., CD31) can bind to the expressed CD38 and activate cytokine production, e.g., increase cytokine release by at least 10%, 20%, 30%, 40%, or 50%. Furthermore, in some embodiments, the animal does not express endogenous CD38 or expresses a reduced level of endogenous CD38 (e.g., less than 50%, less than 40%, less than 30%, less than 20%, less than 10%, or less than 5% as compared to the expression level in an animal without the genetic modification as described herein). As used herein, the term “endogenous CD38” refers to CD38 protein that is expressed from an endogenous CD38 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human CD38 (NP_001766.2) (SEQ ID NO: 4). In some embodiments, the genome comprises a sequence encoding an amino acid sequence that is at least 70%, 75%, 80%, 85%, 90%, 95%, 99%, or 100% identical to SEQ ID NO: 4.

The genome of the genetically modified animal can comprise a replacement at an endogenous CD38 gene locus of a sequence encoding a region of endogenous CD38 with a sequence encoding all or a portion of the extracellular region of human CD38. In some embodiments, the sequence that is replaced is any sequence within the endogenous CD38 gene locus, e.g., exon 1, exon 2, exon 3, exon 4, exon 5, exon 6, exon 7, exon 8, 5′-UTR, 3′-UTR, the first intron, the second intron, and the third intron, the fourth intron, the fifth intron, the sixth intron, the seventh intron, etc. In some embodiments, the sequence that is replaced is within the regulatory region of the endogenous CD38 gene. In some embodiments, the sequence that is replaced is exon 1, exon 2, exon 3, exon 4, exon 5, exon 6, exon 7, exon 8, or a part thereof, of an endogenous mouse CD38 gene locus. In some embodiments, the sequence that is replaced is within exon 1 of an endogenous mouse CD38 gene locus. In some embodiments, the sequence that is replaced starts within exon 2 and ends within exon 3 of the endogenous mouse CD38 gene locus.

The genetically modified animal can have one or more cells expressing a human or chimeric CD38 (e.g., humanized CD38) having a cytoplasmic region, a transmembrane region, and an extracellular region, wherein the extracellular region comprises a sequence that is at least 50%, 60%, 70%, 80%, 90%, 95%, 99% identical to the extracellular region of human CD38. In some embodiments, the extracellular region of the humanized CD38 has a sequence that has at least 10, 20, 30, 40, 50, 60, 70, 80, 90, 100, 110, 120, 130, 140, 150, 160, 170, 180, 190, 200, 210, 220, 230, 240, or 250 amino acids (e.g., contiguously or non-contiguously) that are identical to human CD38. Because human CD38 and non-human CD38 (e.g., mouse CD38) sequences, in many cases, are different, antibodies that bind to human CD38 will not necessarily have the same binding affinity with non-human CD38 or have the same effects to non-human CD38. Therefore, the genetically modified animal having a human or a humanized extracellular region can be used to better evaluate the effects of anti-human CD38 antibodies in an animal model. In some embodiments, the genome of the genetically modified animal comprises a sequence encoding an amino acid sequence that corresponds to part or the entire sequence of exon 2, exon 3, exon 4, exon 5, exon 6, exon 7, and/or exon 8 of human CD38, part or the entire sequence of extracellular region of human CD38, part or the entire sequence of amino acids 43-300 of SEQ ID NO: 4, or part or the entire sequence of amino acids 79-300 of SEQ ID NO: 4.

In some embodiments, the non-human animal can have, at an endogenous CD38 gene locus, a nucleotide sequence encoding a chimeric human/non-human CD38 polypeptide, wherein a human portion of the chimeric human/non-human CD38 polypeptide comprises all or a portion of human CD38 extracellular domain, and wherein the animal expresses a functional CD38 on a surface of a cell of the animal. The human portion of the chimeric human/non-human CD38 polypeptide can comprise a portion of exon 1, exon 2, exon 3, exon 4, exon 5, exon 6, exon 7, and/or exon 8 of human CD38. In some embodiments, the human portion of the chimeric human/non-human CD38 polypeptide can comprise a sequence that is at least 80%, 85%, 90%, 95%, or 99% identical to amino acids 43-300 or 79-300 of SEQ ID NO: 4.

In some embodiments, the non-human portion of the chimeric human/non-human CD38 polypeptide comprises cytoplasmic and/or transmembrane regions of an endogenous non-human CD38 polypeptide. There may be several advantages that are associated with the cytoplasmic and/or transmembrane regions of an endogenous non-human CD38 polypeptide. For example, once a CD38 ligand (e.g., CD31) or an anti-CD38 antibody binds to CD38, they can properly transmit extracellular signals into the cells and initiate the downstream pathway. A human or humanized transmembrane and/or cytoplasmic regions may not function properly in non-human animal cells. In some embodiments, some (e.g., about 1, 2, 3, 4, 5, 6, 7, 8, 9, 10, 15, 20, 25, 30, 35, 36, 37, or 38) extracellular amino acids that are close to the transmembrane region of CD38 are also derived from endogenous sequence. These amino acids can also be important for transmembrane signal transmission.

In some embodiments, a few transmembrane amino acids that are close to the extracellular region of CD38 are deleted.

Furthermore, the genetically modified animal can be heterozygous with respect to the replacement at the endogenous CD38 locus, or homozygous with respect to the replacement at the endogenous CD38 locus.

In some embodiments, the humanized CD38 locus lacks a human CD38 5′-UTR. In some embodiment, the humanized CD38 locus comprises a rodent (e.g., mouse) 5′-UTR. In some embodiments, the humanization comprises a human or mouse 3′-UTR. In appropriate cases, it may be reasonable to presume that the mouse and human CD38 genes appear to be similarly regulated based on the similarity of their 5′-flanking sequence. As shown in the present disclosure, humanized CD38 mice that comprise a replacement at an endogenous mouse CD38 locus, which retain mouse regulatory elements but comprise a humanization of CD38 encoding sequence, do not exhibit pathologies. Both genetically modified mice that are heterozygous or homozygous for humanized CD38 are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized CD38 gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The disclosure also relates to an offspring of the non-human mammal. The present disclosure further relates to a cell or cell line (e.g., stem cell or embryonic stem cell), or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized CD38 in the genome of the animal.

In some embodiments, the non-human mammal comprises the genetic construct as described herein (e.g., gene construct as shown in FIG. 2 or FIG. 13). In some embodiments, a non-human mammal expressing human or humanized CD38 is provided. In some embodiments, the tissue-specific expression of human or humanized CD38 protein is provided.

In some embodiments, the expression of human or humanized CD38 in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human CD38 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized CD38 protein.

In some embodiments, the expression of human or humanized CD38 protein is controllable, as by the addition of a specific inducer or repressor substance. In some embodiments, the specific inducer is selected from Tet-Off System/Tet-On System, or Tamoxifen System.

### Vectors

The present disclosure relates to a targeting vector, comprising: a) a DNA fragment homologous to the 5′ end of a region to be altered (5′ arm), which is selected from the CD38 gene genomic DNAs in the length of 100 to 10,000 nucleotides; b) a desired/donor DNA (e.g, cDNA) or RNA (e.g., mRNA) sequence encoding a donor region; and c) a second DNA fragment homologous to the 3′ end of the region to be altered (3′ arm), which is selected from the CD38 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000071.6; c) the DNA fragment homologous to the 3′ end of the region to be altered (3′ arm) is selected from the nucleotide sequences that have at least 90% homology to the NCBI accession number NC_000071.6.

In some embodiments, a) the DNA fragment homologous to the 5′ end of a region to be altered (5′ arm) is selected from the nucleotides from the position 43864272 to the position 43869001 of the NCBI accession number NC_000071.6; c) the DNA fragment homologous to the 3′ end of the region to be altered (3′ arm) is selected from the nucleotides from the position 43869119 to the position 43872901 of the NCBI accession number NC_000071.6.

In some embodiments, a) the DNA fragment homologous to the 5′ end of a region to be altered (5′ arm) is selected from the nucleotides from the position 43896398 to the position 43900333 of the NCBI accession number NC_000071.6; c) the DNA fragment homologous to the 3′ end of the region to be altered (3′ arm) is selected from the nucleotides from the position 43902059 to the position 43907450 of the NCBI accession number NC_000071.6.

In some embodiments, a) the DNA fragment homologous to the 5′ end of a region to be altered (5′ arm) is selected from the nucleotides from the position 43898964 to the position 43900333 of the NCBI accession number NC_000071.6; c) the DNA fragment homologous to the 3′ end of the region to be altered (3′ arm) is selected from the nucleotides from the position 43902059 to the position 43903453 of the NCBI accession number NC_000071.6.

In some embodiments, the length of the selected genomic nucleotide sequence in the targeting vector can be more than about 500 bp, about 600 bp, about 700 bp, about 800 bp, about 900 bp, about 1 kb, about 2 kb, about 3 kb, about 3.5 kb, about 4 kb, about 4.5 kb, about 5 kb, about 5.5 kb, or about 6 kb. In some embodiments, the desired/donor DNA (e.g, cDNA) or RNA (e.g., mRNA) sequence comprises a sequence from NM_001775.4.

In some embodiments, the region to be altered is exon 1, exon 2, exon 3, exon 4, exon 5, exon 6, exon 7, and/or exon8 of CD38 gene (e.g., exon 1; alternatively, exon 2 and exon 3 of mouse CD38 gene).

The targeting vector can further include a selected gene marker.

In some embodiments, the sequence of the 5′ arm is shown in SEQ ID NO: 52; and the sequence of the 3′ arm is shown in SEQ ID NO: 53. In some embodiments, the sequence of the 5′ arm is shown in SEQ ID NO: 6; and the sequence of the 3′ arm is shown in SEQ ID NO: 7. In some embodiments, the sequence of the 5′ arm is shown in SEQ ID NO: 18; and the sequence of the 3′ arm is shown in SEQ ID NO: 19. In some embodiments, the sequence of the 5′ arm is at least 90%, 95%, or 100% identical to SEQ ID NO: 6, 18, or 52. In some embodiments, the sequence of the 3′ arm is at least 90%, 95%, or 100% identical to SEQ ID NO: 7, 19, or 53.

In some embodiments, the desired/donor sequence comprises a sequence that is derived from human CD38 (e.g., 214-990 of NM_001775.4; or 322-990 of NM_001775.4). For example, the target region in the targeting vector is a part or entirety of the nucleotide sequence of a human CD38, preferably exon 1, exon 2, exon 3, exon 4, exon 5, exon 6, exon 7, and/or exon 8 of the human CD38. In some embodiments, the nucleotide sequence of the humanized CD38 encodes the entire or the part of human CD38 protein with the NCBI accession number NP_001766.2 (SEQ ID NO: 4). In some embodiments, the target region is an mRNA sequence (e.g., 214-990 of NM_001775.4; or 322-990 of NM_001775.4), or a cDNA sequence derived thereof. In some embodiments, the target region comprises a DNA sequence that is at least 50%, 60%, 70%, 80%, 90%, 99%, or 100% identical to SEQ ID NO: 8 or SEQ ID NO: 54. In some embodiments, the target region comprises at least or about 1, 2, 3, 4, 5, 6, 7, 8, 9, 10, 20, 30, 40, 50, 60, 70, 80, 90, 100, or 110 nucleotides (contiguous or non-contiguous) of 214-321 of SEQ ID NO: 3.

In some embodiments, the target region as described herein are operably linked to a Woodchuck Hepatitis Virus (WHP) Posttranscriptional Regulatory Element (WPRE) and/or a polyA (polyadenylation) signal sequence. The WPRE element is a DNA sequence that, when transcribed, creates a tertiary structure enhancing expression. The sequence can be used to increase expression of genes delivered by viral vectors. WPRE is a tripartite regulatory element with gamma, alpha, and beta components. In some embodiments, the sequence comprising the WPRE and polyA sequence is at least 80%, 85%, 90%, 95%, 96%, 97%, 98%, 99%, or 100% identical to nucleic acids 778-1594 of SEQ ID NO: 51.

In some embodiments, the target region as described herein are operably linked to an endogenous 3′UTR (e.g., mouse 3′UTR) and/or a LoxP STOP sequence. In some embodiments, the LoxP STOP sequence is at least 80%, 85%, 90%, 95%, 96%, 97%, 98%, 99%, or 100% identical to SEQ ID NO: 5.

The disclosure also provides vectors for constructing a humanized animal model or a knock-out model. In some embodiments, the vectors comprise sgRNA sequence, wherein the sgRNA sequence target CD38 gene, and the sgRNA is unique on the target sequence of the gene to be altered, and meets the sequence arrangement rule of 5′-NNN (20)-NGG3′ or 5′-CCN-N (20)-3′; and in some embodiments, the targeting site of the sgRNA in the mouse CD38 gene is located on exon 1, exon 2, exon 3, exon 4, exon 5, exon 6, exon 7, exon 8, intron 1, intron 2, intron 3, intron 4, intron 5, intron 6, intron 7, upstream of exon 1, or downstream of exon 8 of the mouse CD38 gene. In some embodiments, the targeting site of the sgRNA in the mouse CD38 gene is located on exon 2, intron 2, and/or intron 3 of the mouse CD38 gene.

In some embodiments, the 5′ targeting sequence is shown as SEQ ID NOS: 20-27, and the sgRNA sequence recognizes the 5′ targeting site. In some embodiments, the 3′ targeting sequence is shown as SEQ ID NOS: 28-34 and the sgRNA sequence recognizes the 3′ targeting site. Thus, the disclosure provides sgRNA sequences for constructing a genetic modified animal model. In some embodiments, the oligonucleotide sgRNA sequences are set forth in SEQ ID NOS: 35-42.

In some embodiments, the 5′ targeting sequence is shown as SEQ ID NO: 21 and the 3′ targeting sequence is shown as SEQ ID NO: 30.

In some embodiments, the disclosure relates to a plasmid construct (e.g., pT7-sgRNA) including the sgRNA sequence, and/or a cell including the construct.

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous CD38 gene locus, a sequence encoding a region of an endogenous CD38 with a sequence encoding all or a portion of the extracellular region of human or chimeric CD38. In some embodiments, the replacement occurs in a germ cell, a somatic cell, a blastocyst, or a fibroblast, etc. The nucleus of a somatic cell or the fibroblast can be inserted into an enucleated oocyte.

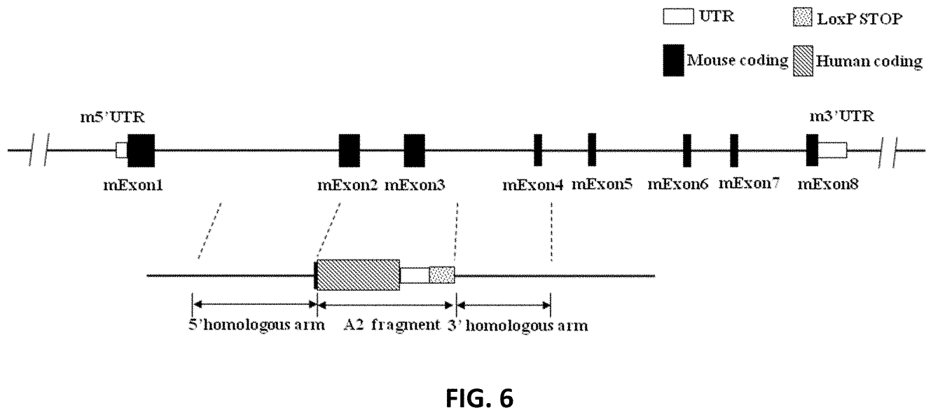

FIG. 6 shows a humanization strategy for a mouse CD38 locus. In FIG. 6, the targeting strategy involves a vector comprising the 5′ end homologous arm, the fragment comprising a human CD38 coding sequence, and 3′ homologous arm. The process can involve replacing endogenous CD38 sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous CD38 sequence with human CD38 sequence.

Thus, in some embodiments, the methods for making a genetically modified, humanized animal, can include the step of replacing at an endogenous CD38 locus (or site), a nucleic acid encoding a sequence encoding a region of endogenous CD38 with a sequence encoding all or a portion of the extracellular region of human CD38. The sequence can include a region (e.g., a part or the entire region) of exon 1, exon 2, exon 3, exon 4, exon 5, exon 6, exon 7, and/or exon 8 of a human CD38 gene, a human CD38 transcript, or a cDNA sequence derived thereof. In some embodiments, the sequence includes a region of exon 1, exon 2, exon 3, exon 4, exon 5, exon 6, exon 7, and/or exon 8 of a human CD38 gene (e.g., amino acids 43-300 or 79-300 of SEQ ID NO: 4). In some embodiments, the region is located within the extracellular region of human CD38. In some embodiments, the endogenous CD38 locus is exon 1, exon 2, exon 3, exon 4, exon 5, exon 6, exon 7, and/or exon 8 of mouse CD38 gene (e.g., a region within exon 1; alternatively exon 2 and exon 3 of mouse CD38 gene).

In some embodiments, the methods of modifying a CD38 locus of a mouse to express a chimeric human/mouse CD38 peptide can include the steps of replacing at the endogenous mouse CD38 locus a nucleotide sequence encoding a mouse CD38 with a nucleotide sequence encoding a human CD38, thereby generating a sequence encoding a chimeric human/mouse CD38.

In some embodiments, the nucleotide sequence encoding the chimeric human/mouse CD38 can include a first nucleotide sequence encoding a cytoplasmic and transmembrane region of a mouse CD38; a second nucleotide sequence encoding an extracellular region of mouse CD38; and a third nucleotide sequence encoding an extracellular region of human CD38.

In some embodiments, the nucleotide sequence encoding the chimeric human/mouse CD38 can include a first nucleotide sequence encoding a cytoplasmic and transmembrane region of a mouse CD38; and a second nucleotide sequence encoding an extracellular region of human CD38.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a CD38 gene humanized animal model, involving the following steps:

(a) providing the cell (e.g. a fertilized egg cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized CD38 protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized CD38, which are useful for testing agents that can decrease or block the interaction between CD38 and CD38 ligands (e.g., CD31) or the interaction between CD38 and anti-human CD38 antibodies, testing whether an agent can increase or decrease the immune response, and/or determining whether an agent is an CD38 agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In some embodiments, the genetically modified animals can be used for determining effectiveness of an anti-CD38 antibody for the treatment of cancer. The methods involve administering the anti-CD38 antibody (e.g., anti-human CD38 antibody) to the animal as described herein, wherein the animal has a tumor; and determining the inhibitory effects of the anti-CD38 antibody to the tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MM or CT.

In some embodiments, the tumor comprises one or more cancer cells (e.g., human or mouse cancer cells) that are injected into the animal. In some embodiments, the anti-CD38 antibody prevents a CD38 ligand (e.g., CD31) from binding to CD38. In some embodiments, the anti-CD38 antibody does not prevent a CD38 ligand (e.g., CD31) from binding to CD38.

In some embodiments, the genetically modified animals can be used for determining whether an anti-CD38 antibody is a CD38 agonist or antagonist. In some embodiments, the methods as described herein are also designed to determine the effects of the agent (e.g., anti-CD38 antibodies) on CD38, e.g., whether the agent can stimulate immune cells or inhibit immune cells, whether the agent can increase or decrease the production of cytokines, whether the agent can activate or deactivate immune cells, whether the agent can upregulate the immune response or downregulate immune response, and/or whether the agent can induce complement-dependent cytotoxicity (CDC) or antibody-dependent cellular cytoxicity (ADCC). In some embodiments, the genetically modified animals can be used for determining the effective dosage of a therapeutic agent for treating a disease in the subject, e.g., cancer, or autoimmune diseases.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the anti-CD38 antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer as described herein is Lymphoma, B cell tumor, T cell tumor, bone marrow/monocyte tumor, non-small cell lung cancer, leukemia, ovarian cancer, nasopharyngeal cancer, breast cancer, endometrial cancer, colon cancer, rectal cancer, stomach cancer, bladder cancer, Lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, Myelodysplastic Syndrome, and Sarcoma. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T cell lymphoma, and Waldenstrom macroglobulinemia. In some embodiments, the sarcoma is selected from osteosarcoma, Ewing sarcoma, leiomyosarcoma, synovial sarcoma, soft tissue sarcoma, angiosarcoma, liposarcoma, fibrosarcoma, rhabdomyosarcoma Chondrosarcoma. In some embodiments, the cancer as described herein is B cell tumors, T cell tumors, bone marrow/monocyte tumors. In some embodiments, the cancer as described herein is B or T cell acute lymphoblastic leukemia (ALL), acute myeloid leukemia (AML), non-Hodgkin's lymphoma (NHL) and multiple myeloma (MM), nasopharyngeal carcinoma, or lung cancer.

In some embodiments, the anti-CD38 antibody is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the anti-CD38 antibody is designed for treating hepatocellular, ovarian, colon, or cervical carcinomas. In some embodiments, the anti-CD38 antibody is designed for treating advanced breast cancer, advanced ovarian cancer, and/or advanced refractory solid tumor. In some embodiments, the anti-CD38 antibody is designed for treating metastatic solid tumors, NSCLC, melanoma, non-Hodgkin lymphoma, colorectal cancer, and multiple myeloma. In some embodiments, the anti-CD38 antibody is designed for treating melanoma, pancreatic carcinoma, mesothelioma, hematological malignancies (e.g., Non-Hodgkin's lymphoma, lymphoma, chronic lymphocytic leukemia), or solid tumors (e.g., advanced solid tumors). In some embodiments, the anti-CD38 antibody is designed for treating carcinomas (e.g., nasopharynx carcinoma, bladder carcinoma, cervix carcinoma, kidney carcinoma or ovary carcinoma).

In some embodiments, the anti-CD38 antibody is designed for treating various allergic disorders (e.g., asthma). In some embodiments, the anti-CD38 antibody is designed for treating various immune system disorders, e.g., allergy, asthma, myocarditis, nephritis, hepatitis, systemic lupus erythematosus, rheumatoid arthritis, scleroderma, hyperthyroidism, primary thrombocytopenic purpura, autoimmune hemolytic anemia, ulcerative colitis, self-immune liver disease, diabetes, pain, or neurological disorders, etc. In some embodiments, the immune system disorder is rheumatoid arthritis. In some embodiments, the anti-CD38 antibody is designed for treating various autoimmune disorders (e.g., rheumatoid arthritis, inflammatory bowel disease, systemic lupus erythematosus, or multiple sclerosis). In some embodiments, the anti-CD38 antibody is designed for reducing inflammation. In some embodiments, the inflammation includes, but not limited to: chronic inflammation, degenerative inflammation, exudative inflammation (serous inflammation, fibrinitis, suppurative inflammation, hemorrhagic inflammation, necrotitis, catarrhal inflammation), proliferative inflammation, specific inflammation (tuberculosis, syphilis, Leprosy, lymphogranuloma, etc.). Thus, the methods as described herein can be used to determine the effectiveness of an anti-CD38 antibody in inhibiting immune response. In some embodiments, the anti-CD38 antibody is designed for treating various cardiovascular diseases.

The present disclosure also provides methods of screening CD38-specific immunomodulators. The methods involve administering the immunomodulator to the animal as described herein, wherein the animal has a tumor; and determining the inhibitory effects (e.g., any of the inhibitory effects described herein) of the immunomodulator to the tumor. In some embodiments, the immunomodulatory is a drug (e.g., antibody) or CAR-T.

The present disclosure also provides methods of determining toxicity of an antibody (e.g., anti-CD38 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the CD38 gene function, human CD38 antibodies, drugs for human CD38 targeting sites, the drugs or efficacies for human CD38 targeting sites, the drugs for immune-related diseases and antitumor drugs.

Genetically Modified Animal Model with Two or More Human or Chimeric Genes

The present disclosure further relates to methods for generating genetically modified animal model with two or more human or chimeric genes. The animal can comprise a human or chimeric CD38 gene and a sequence encoding an additional human or chimeric protein.

In some embodiments, the additional human or chimeric protein can be CD31, CD3, B-cell maturation antigen (BCMA), interleukin-15 receptor (IL15R), adenosine A2a receptor (A2aR), programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (TNFRSF4 or OX40).

The methods of generating genetically modified animal model with two or more human or chimeric genes (e.g., humanized genes) can include the following steps:

(a) using the methods of introducing human CD38 gene or chimeric CD38 gene as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric CD31, CD3, BCMA, IL15R, A2aR, PD-1, CTLA-4, LAG-3, BTLA, PD-L1, CD27, CD28, CD47, CD137, CD154, TIGIT, TIM-3, GITR, SIRPα, or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the CD38 humanization is directly performed on a genetically modified animal having a human or chimeric CD31, CD3, BCMA, IL15R, A2aR, PD-1, CTLA-4, BTLA, PD-L1, CD27, CD28, CD47, CD137, CD154, TIGIT, TIM-3, GITR, SIRPα, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-CD38 antibody and an additional therapeutic agent for the treatment of cancer. The methods include administering the anti-CD38 antibody and the additional therapeutic agent to the animal, wherein the animal has a tumor; and determining the inhibitory effects of the combined treatment to the tumor. In some embodiments, the additional therapeutic agent is an antibody that specifically binds to CD31, CD3, BCMA, IL15R, A2aR, PD-1, CTLA-4, BTLA, PD-L1, CD27, CD28, CD47, CD137, CD154, TIGIT, TIM-3, GITR, SIRPα, or OX40. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancer as described herein, e.g., melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, the combination treatment is designed for treating metastatic solid tumors, NSCLC, melanoma, B-cell non-Hodgkin lymphoma, colorectal cancer, and multiple myeloma. In some embodiments, the combination treatment is designed for treating melanoma, carcinomas (e.g., pancreatic carcinoma), mesothelioma, hematological malignancies (e.g., Non-Hodgkin's lymphoma, lymphoma, chronic lymphocytic leukemia), or solid tumors (e.g., advanced solid tumors).

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

Attune™ Nxt Acoustic Focusing Cytometer was purchased from Thermo Fisher Scientific (Model: Attune™ Nxt).

Low speed refrigerated centrifuge was purchased from Beijing Baiyang Medical Equipment Co., Ltd. (Model: BY-R320).

Heraeus™ Fresco™ 21 Microcentrifuge was purchased from Thermo Fisher Scientific (Model: Fresco™ 21).

BioTek Epoch™ Microplate Reader was purchased from BioTeK Instruments, Inc. (Model: Epoch™)

PerCP/Cy5.5 anti-mouse TCR β chain was purchased from BioLegend (Catalog number: 109228).

FITC anti-mouse CD19 Antibody (mCD19-FITC-A) was purchased from BioLegend (Catalog number: 115506).

Brilliant Violet 421™ anti-mouse CD38 Antibody (mCD38-BV421-A) was purchased from BioLegend (Catalog number: 102732).

APC mouse anti-human CD38 Antibody (hCD38-APC-A) was purchased from BioLegend (Catalog number: 356606).

Human CD38 PE-conjugated Antibody was purchased from R&D Systems, Inc. (Catalog number: FAB2404P).

Brilliant Violet 510™ anti-mouse CD45 Antibody was purchased from BioLegend (Catalog number: 103138).

EcoRI, BamHI, StuI, and BglII restriction enzymes were purchased from NEB (Catalog numbers: R0101M, R0136M, R0187M, and R0144M, respectively).

### Example 1: Mice with Humanized CD38 Gene

In this example, a non-human animal (e.g., a mouse) was modified to include a nucleotide sequence encoding human CD38 protein, and the obtained genetically-modified non-human animal can express a human or humanized CD38 protein in vivo. The mouse CD38 gene (NCBI Gene ID: 12494, Primary source: MGI: 107474, UniProt ID: P56528) is located at 43868809 to 43912374 of chromosome 5 (NC_000071.6), and the human CD38 gene (NCBI Gene ID: 952, Primary source: HGNC:1667, UniProt ID: P28907) is located at 15778328 to 15853232 of chromosome 4 (NC_000004.12). The mouse CD38 transcript sequence NM_007646.5 is set forth in SEQ ID NO: 1, and the corresponding protein sequence NP_031672.2 is set forth in SEQ ID NO: 2. The human CD38 transcript sequence NM_001775.4 is set forth in SEQ ID NO: 3, and the corresponding protein sequence NP_001766.2 is set forth in SEQ ID NO: 4. Mouse and human CD38 gene loci are shown in FIG. 1A and FIG. 1B, respectively.

A nucleotide sequence encoding human CD38 protein was introduced into the endogenous mouse CD38 locus, such that the mouse can express a human or humanized CD38 protein. Mouse cells can be modified by various gene-editing techniques, for example, replacement of specific mouse CD38 gene sequences with human CD38 gene sequences at the endogenous mouse CD38 locus. For example, under control of a mouse CD38 regulatory element, a 117 bp sequence containing at least a portion of exon 1 of the mouse CD38 gene can be replaced with the coding sequence of human CD38 extracellular region. Meanwhile, an auxiliary sequence WPRE (Woodchuck hepatitis B virus post-transcriptional regulatory element) and polyA (polyadenylation) can be inserted after the coding sequence of the extracellular region of human CD38, to obtain a mouse chimeric CD38 locus as shown in FIG. 13, thereby humanizing mouse CD38 gene. The DNA sequence of the humanized mouse CD38 gene (chimeric CD38 gene DNA) is shown in SEQ ID NO: 51.

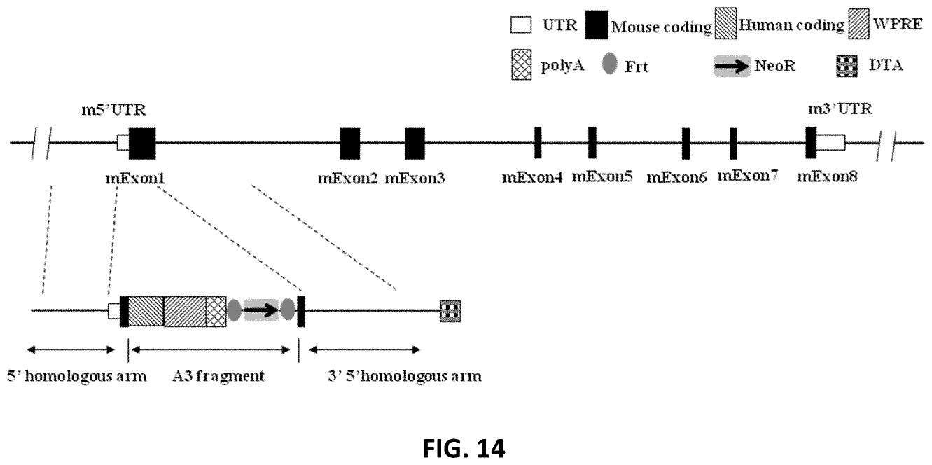

As shown in the schematic diagram of the targeting strategy in FIG. 14, the targeting vector has homologous arm sequences upstream and downstream of mouse CD38 gene locus, and an “A3 fragment” comprising the human CD38 protein coding sequence and the auxiliary sequence WPRE-polyA. Specifically, the upstream homologous arm sequence (5′ homologous arm, SEQ ID NO: 52) is identical to nucleotide sequence of 43864272-43869001 of NCBI accession number NC_000071.6, and the downstream homologous arm sequence (3′ homologous arm, SEQ ID NO: 53) is identical to nucleotide sequence of 43869119-43872901 of NCBI accession number NC_000071.6. Some features of the A3 fragment are as follows: the A3 fragment is located within exon 1 of the mouse CD38 gene; the A3 fragment comprises the human CD38 coding sequence (SEQ ID NO: 54, which is identical to the nucleotide sequence 214-990 of NCBI accession number NM_001775.4), the auxiliary sequence WPRE-polyA, and a Neo cassette composed of the neomycin phosphotransferase coding sequence Neo and two sets of site-specific recombination systems Frt.

The connection between the upstream of the human CD38 gene sequence and the mouse CD38 gene locus was designed as:

(SEQ ID NO: 55)
5′-CTCCTGGTCCTGATCGCCTTGGTAGTAGGGATCGTGGTCgtcccg
aggtggcgccagcagtggagcggtccgggcaccac-3′,

wherein the “C” of sequence “TGGTC” is the last nucleotide of the mouse sequence, and the “g” of sequence “gtccc” is the first nucleotide of the human sequence. The connection between the downstream of the Neo cassette and the mouse CD38 gene locus was designed as:

(SEQ ID NO: 56)
5′-GAACTTCATCAGTCAGGTACATAATGGTGGATCCCCATGGAGGTGA
GTTGGCTTCTGAGGCTCACTCTAGGCACAGTGCG-3′,

wherein the last “G” of sequence “CATGG” is the last nucleotide of the Neo cassette, and the “A” of sequence “AGGTG” is the first nucleotide of the mouse sequence. In addition, a coding gene with a negative selectable marker (a gene encoding diphtheria toxin A subunit (DTA)) was also inserted downstream of the 3′ homologous arm of the targeting vector. The mRNA sequence of the engineered mouse CD38 after humanization and its encoded protein sequence are shown in SEQ ID NO: 57 and SEQ ID NO: 58, respectively.

Alternatively, a 1725 bp sequence comprising at least exon 2 and exon 3 of the mouse CD38 gene can be replaced with a human gene sequence, and a LoxP STOP sequence (SEQ ID NO: 5) can be inserted after the mouse 3′UTR sequence, thereby humanizing mouse CD38 gene. The schematic diagram of the chimeric CD38 locus is shown in FIG. 2.

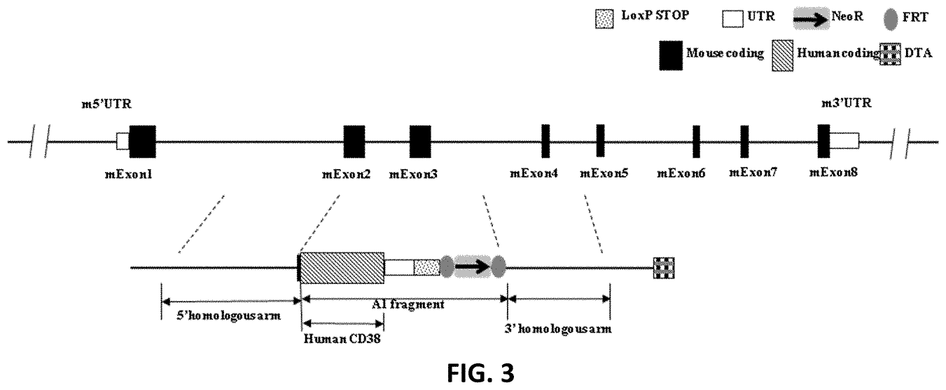

As shown in the schematic diagram of the targeting strategy in FIG. 3, the targeting vector has an upstream homologous arm sequence, a downstream homologous arm sequence, and an “A1 fragment” comprising a nucleotide sequence encoding the human CD38 protein. Specifically, the upstream homologous arm sequence (5′ homologous arm, SEQ ID NO: 6) is identical to nucleotide sequence of 43896398-43900333 of NCBI accession number NC_000071.6, and the downstream homologous arm sequence (3′ homologous arm, SEQ ID NO: 7) is identical to nucleotide sequence of 43902059-43907450 of NCBI accession number NC_000071.6. Some features of the A1 fragment are as follows: the A1 fragment comprises a human CD38 coding sequence (SEQ ID NO: 8, which is identical to the nucleotide sequence 322-990 of NCBI accession number NM_001775.4), 3′UTR sequence of mouse CD38 gene, the LoxP STOP sequence, and a Neo cassette.

The connection between the upstream of the human CD38 gene sequence and the mouse CD38 gene locus was designed as:

(SEQ ID NO: 9)
5′-aatatttttaaaatctgttttccatctttcattttatagaCATGTA
GACTGCCAAAGTGTATGGGATGCTTTCAAGGGTG-3′,

wherein the last “a” of sequence “ataga” is the last nucleotide of the mouse sequence, and the “C” of sequence “CATGT” is the first nucleotide of the human sequence. The downstream of the human CD38 sequence was directly connected to the upstream of the mouse 3′UTR. The connection between the downstream of the mouse 3′UTR and the upstream of the LoxP STOP sequence was designed as: 5′-gatggttcagtccattaaatgatactttgtaagtGTCGACattaagggttccggatcctcggggacaccaaatat-3′ (SEQ ID NO: 50), wherein the last “t” of sequence “taagt” is the last nucleotide of the mouse 3′UTR sequence, and the first “a” of sequence “attaa” is the first nucleotide of the LoxP STOP sequence. The connection between the upstream of the Neo cassette and the downstream of the loxP STOP sequence was designed as:

(SEQ ID NO: 10)
5′-gacatggtaagtaagcttgggctgcaggtcgagggacctaGAATTC
CGAAGTTCCTATTCTCTAGAAAGTATAGGAACTT-3′,

wherein the last “a” of sequence “accta” is the last nucleotide of the loxP STOP sequence, and the “G” of sequence “GAATT” is the first nucleotide of the Neo cassette. The connection between the downstream of the Neo cassette and the mouse sequence was designed as:

(SEQ ID NO: 11)
5′-GTATAGGAACTTCATCAGTCAGGTACATAATGGTGGATCCtaagac
atccccaaatccagtctctccctcactttccctc-3′ ,

wherein the last “C” of sequence “GATCC” is the last nucleotide of the Neo cassette, and the “t” of sequence “taaga” is the first nucleotide of the mouse sequence. In addition, a coding gene with a negative selectable marker (a gene encoding diphtheria toxin A subunit (DTA)) was also inserted downstream of the 3′ homologous arm of the targeting vector. The mRNA sequence of the engineered mouse CD38 after humanization and its encoded protein sequence are shown in SEQ ID NO: 12 and SEQ ID NO: 13, respectively.

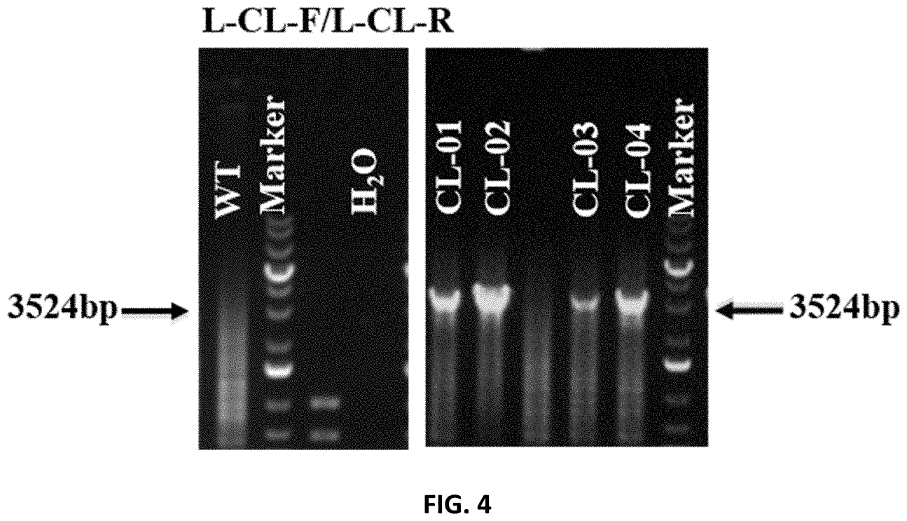

The targeting vector was constructed, e.g., by restriction enzyme digestion and ligation, or synthesized directly. The constructed targeting vector sequence was preliminarily verified by restriction enzyme digestion, then verified by sequencing. The correct targeting vector was electroporated and transfected into embryonic stem cells of C57BL/6 mice. The positive selectable marker gene was used to screen the cells, and the integration of exogenous genes was confirmed by PCR and Southern Blot. Correct positive clone cells were screened. Exemplary results of PCR identification are shown in FIG. 4, in which 4 clones numbered CL-01 to CL-04 were identified as positive clones.

The following primers were used in PCR:

L-CL-F:
(SEQ ID NO: 14)
5′-ATTCTCTGCAAATTACCAACTCTTCCA-3′
L-CL-R:
(SEQ ID NO: 15)
5′-GTTACATCGAAAGCAGGGCTCAGG-3′

The primer L-CL-F is located on the 5′ homology arm, and L-CL-R is located on the 3′UTR sequence of the mouse CD38 gene.

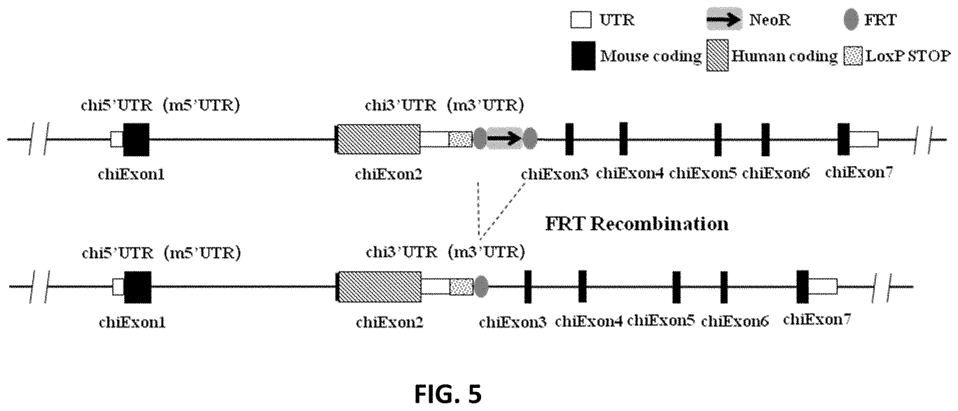

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp mice to remove the positive selectable marker gene (schematic diagram of the process is shown in FIG. 5), and then the humanized CD38 homozygous mice were obtained by breeding with each other.

In addition, CRISPR/Cas gene editing technology was also used to obtain the CD38 gene humanized mice. For example, to generate the CD38 gene humanized mice with the humanized mouse CD38 gene locus shown in FIG. 2, a targeting strategy was designed as shown in FIG. 6. The targeting vector has an upstream homologous arm sequence (5′ homologous arm; SEQ ID NO: 18), a downstream homologous arm sequence (3′ homologous arm; SEQ ID NO: 19), and an “A2 fragment” comprising a nucleotide sequence encoding human CD38 protein. Specifically, the 5′ homologous arm is identical to nucleotide sequence of 43898964-43900333 of NCBI accession number NC_000071.6, and the 3′ homologous arm is identical to nucleotide sequence of 43902059-43903453 of NCBI accession number NC_000071.6. The difference between the A2 fragment of the targeting vector and the A1 fragment in FIG. 3 is that the A2 fragment does not contain the Neo cassette sequence.

The targeting vector was constructed, e.g., by restriction enzyme digestion and ligation, or synthesized directly. The constructed targeting vector sequence was preliminarily verified by restriction enzyme digestion, then verified by sequencing. The correct targeting vector verified by sequencing was used for subsequence experiments.

Specific sgRNA sequences were designed and synthesized that recognize the 5′ end targeting sites (sgRNA1-sgRNA8) and 3′ end targeting sites (sgRNA9-sgRNA15). The 5′ end targeting sites are located on exon 2, or introns 2-3 (specifically, sgRNAs 1-3, 5, and 7-8 target exon 2; and sgRNAs 4 and 6 target introns 2-3); and the 3′ end targeting site are located on introns 3-4 of the mouse CD38 gene. The targeting site sequence of each sgRNA on the CD38 gene locus is as follows:

sgRNA 1 targeting site (SEQ ID NO: 20):
5′-TGAGTGACCAATTTAACAAGTGG-3′
sgRNA 2 targeting site (SEQ ID NO: 21):
5′-TGAATGTACTCAGTATCTCCTGG-3′
sgRNA 3 targeting site (SEQ ID NO: 22):
5′-TGTGATGTTGCAAGGGTTCTTGG-3′
sgRNA 4 targeting site (SEQ ID NO: 23):
5′-GCCCTCATTACCTTGTTACATGG-3′
sgRNA 5 targeting site (SEQ ID NO: 24):
5′-GAGTGACCAATTTAACAAGTGGG-3′
sgRNA 6 targeting site (SEQ ID NO: 25):
5′-TCAAACCATACCATGTAACAAGG-3′
sgRNA 7 targeting site (SEQ ID NO: 26):
5′-GAGATACTGAGTACATTCAAAGG-3′
sgRNA 8 targeting site (SEQ ID NO: 27):
5′-TCTTCTCTTGTGATGTTGCAAGG-3′
sgRNA 9 targeting site (SEQ ID NO: 28):
5′-AGGGAATTTACCCCCATGAATGG-3′
sgRNA 10 targeting site (SEQ ID NO: 29):
5′-ATGAGCTCAACTCCATTTAGAGG-3′
sgRNA 11 targeting site (SEQ ID NO: 30):
5′-GCTACTTTATAAGGCTGTTGAGG-3′
sgRNA 12 targeting site (SEQ ID NO: 31):
5′-TAAATGGAGTTGAGCTCATGAGG-3′
sgRNA 13 targeting site (SEQ ID NO: 32):
5′-CTAGATTAGTGATCACAAAAAGG-3′
sgRNA 14 targeting site (SEQ ID NO: 33):
5′-ATTCAGCTTAATGGGAACATTGG-3′
sgRNA 15 targeting site (SEQ ID NO: 34):
5′-GGATTAAAAATCCATTCATGGGG-3′

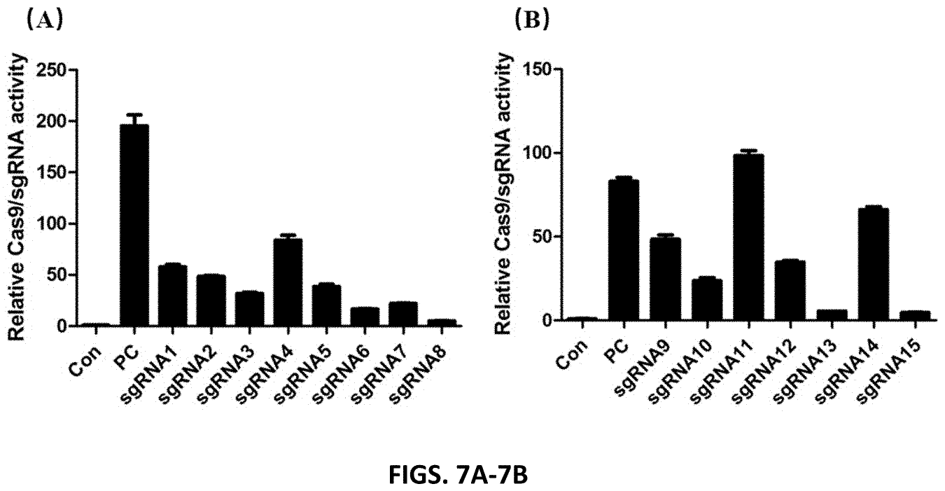

The UCA kit was used to detect the activities of sgRNAs. As shown in FIGS. 7A-7B and Table 3 below, the results showed that the sgRNAs had different activities. In particular, sgRNA8, sgRNA13, and sgRNA15 exhibited relatively low activities, which may be caused by sequence variations of their targeting sites. However, the relative activities of sgRNA8, sgRNA13, and sgRNA15 were still significantly higher than that of the negative control (Con). It is therefore concluded that sgRNA8, sgRNA13 and sgRNA15 can suffice the requirement for gene editing experiment. sgRNA2 and sgRNA11 were randomly selected for subsequent experiments. Oligonucleotides were added to the 5′ end and a complementary strand to obtain a forward oligonucleotide and a reverse oligonucleotide (see Table 4 for the sequences). After annealing, the products were ligated to the pT7-sgRNA plasmid (the plasmid was first linearized with BbsI), respectively, to obtain expression vectors PT7-sgRNA2 and pT7-sgRNA11.

TABLE 3
UCA detection results

| 5′ end targeting site detection result | | 3′ end targeting site detection result | |
|---|---|---|---|
| Con. | 1.00 ± 0.09 | Con. | 1.00 ± 0.03 |
| PC | 195.62 ± 10.45 | PC | 83.06 ± 2.20 |
| sgRNA1 | 57.88 ± 2.26 | sgRNA9 | 48.41 ± 2.72 |
| sgRNA2 | 48.36 ± 1.12 | sgRNA10 | 23.67 ± 1.92 |
| sgRNA3 | 31.69 ± 1.39 | sgRNA11 | 98.48 ± 2.92 |
| sgRNA4 | 83.77 ± 4.87 | sgRNA12 | 34.81±1.05 |
| sgRNA5 | 38.72 ± 2.27 | sgRNA13 | 5.53 ± 0.07 |
| sgRNA6 | 16.49 ± 0.69 | sgRNA14 | 66.15 ± 1.73 |
| sgRNA7 | 22.05 ± 0.36 | sgRNA15 | 4.71 ± 0.23 |
| sgRNA8 | 5.18 ± 0.44 | — | — |

TABLE 4
sgRNA2 sequence

| | |
|---|---|
| SEQID NO: 35 | Upstream: 5′-TGAATGTACTCAGTATCTCC-3′ |
| SEQ ID NO: 36 (forward oligonucleotide) | Upstream: 5′-TAGGTGAATGTACTCAGTATCTCC-3′ |
| SEQ ID NO: 37 | Downstream: 5′-GGAGATACTGAGTACATTCA-3′ |
| SEQ ID NO: 38 (reverse oligonucleotide) | Downstream: 5′-AAACGGAGATACTGAGTACATTCA-3′ |

sgRNA11 sequence

| | |
|---|---|
| SEQ ID NO: 39 | Upstream: 5′-GCTACTTTATAAGGCTGTTG-3′ |
| SEQ ID NO: 40 (forward oligonucleotide) | Upstream: 5′-TAGGCTACTTTATAAGGCTGTTG-3′ |
| SEQ ID NO: 41 | Downstream: 5′-CAACAGCCTTATAAAGTAG-3′ |
| SEQ ID NO: 42 (reverse oligonucleotide) | Downstream: 5′-AAACCAACAGCCTTATAAAGTAG-3′ |

The pT7-sgRNA vector was synthesized, which included a DNA fragment containing the T7 promoter and sgRNA scaffold (SEQ ID NO: 43), and was ligated to the backbone vector (Takara, Catalog number: 3299) after restriction enzyme digestion (EcoRI and BamHI). The resulting plasmid was confirmed by sequencing.

The pre-mixed Cas9 mRNA, the targeting vector, and in vitro transcription products of the pT7-sgRNA2, pT7-sgRNA11 plasmids (using Ambion in vitro transcription kit to carry out the transcription according to the method provided in the product instruction) were injected into the cytoplasm or nucleus of mouse fertilized eggs with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2006. The injected fertilized eggs were then transferred to a culture medium to culture for a short time and then was transplanted into the oviduct of the recipient mouse to produce the genetically modified mice (F0 generation). The mouse population was further expanded by cross-breeding and self-breeding to establish stable homozygous mouse lines with genetically-modified CD38 gene locus.

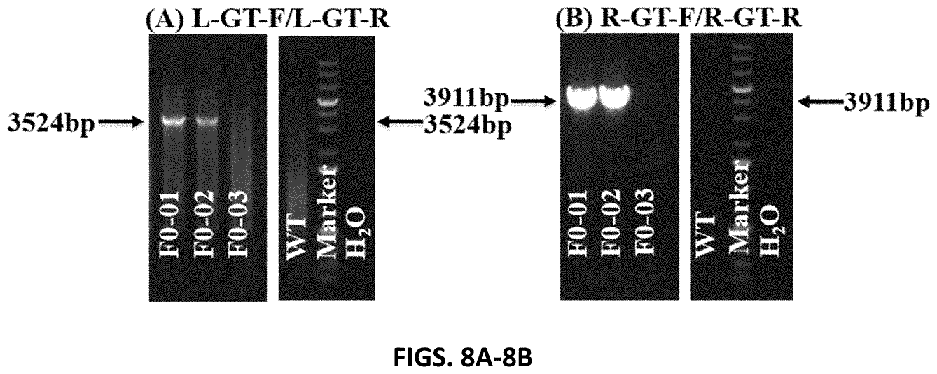

The genotype of somatic cells of F0 generation mice can be identified, e.g., by PCR analysis. The identification results of some F0 generation mice are shown in FIGS. 8A-8B. In view of the 5′ end primer detection result and the 3′ end primer detection result, the 2 mice numbered F0-01 and F0-02 were both identified as positive mice.

The following primers were used in the PCR:

5′ end primers:
L-GT-F (SEQ ID NO: 14):
5′-ATTCTCTGCAAATTACCAACTCTTCCA-3′
L-GT-R (SEQ ID NO: 15):
5′-GTTACATCGAAAGCAGGGCTCAGG-3′
3′ end primers:
R-GT-F (SEQ ID NO: 16):
5′-ATCAGTCTTGCTCAGAATCACTGGTT-3′
R-GT-R (SEQ ID NO: 17):
5′-GGTTGTTGGGACAGTTTTCACTCCA-3′

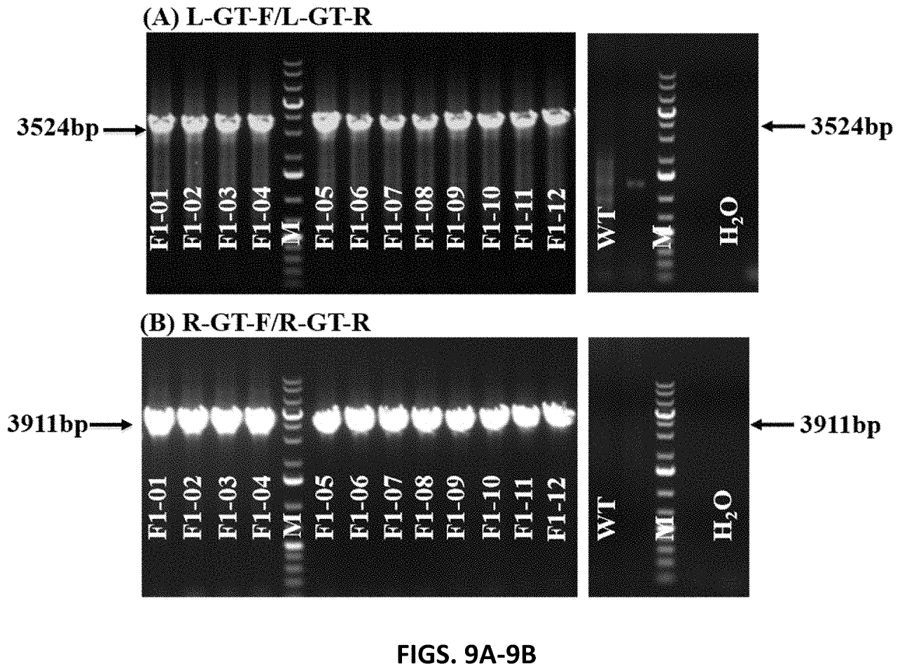

The positive F0 generation CD38 gene humanized mice generated using the targeting strategy shown in FIG. 3 were bred with wild-type mice to generate F1 generation mice. The same method (e.g., PCR) was used for genetic identification of the F1 generation mice. As shown in FIGS. 9A-9B, the 12 mice numbered F1-01 to F1-12 were all identified as positive mice. The F1 generation mice were further analyzed by Southern Blot (See Table 5 for the length of specific probes and target fragments), to confirm whether random insertions were introduced. Specifically, mouse tail genomic DNA was extracted, digested with StuI or BglII restriction enzyme, transferred to a membrane, and then hybridized with probes. Probes P1 and P2 are located upstream of the 5′ homologous arm and on the LoxP STOP sequence, respectively.

TABLE 5
The length of the specific probes and target fragments

| Restriction | | Wild-type | Recombinant sequence |
|---|---|---|---|
| Enzyme | Probe | fragment size | fragment size |
| Stul | P1 | 7.9 kb | 5.4 kb |
| BgIll | P2 | — | 3.4 kb |

The following primers were used to synthesize probes used in Southern Blot assays:

P1-F (SEQ ID NO: 44):
5′-CTAGGCACTTAGCAGGATGCCCTTG-3′,
P1-R (SEQ ID NO: 45):
5′-CCTGAAGCCCAAGGATGTGAAAGGA-3′
P2-F (SEQ ID NO: 46):
5′-AACTGATGAATGGGAGCAGTGGT-3′,
P2-R (SEQ ID NO: 47):
5′-GCAGACACTCTATGCCTGTGTGG-3′;

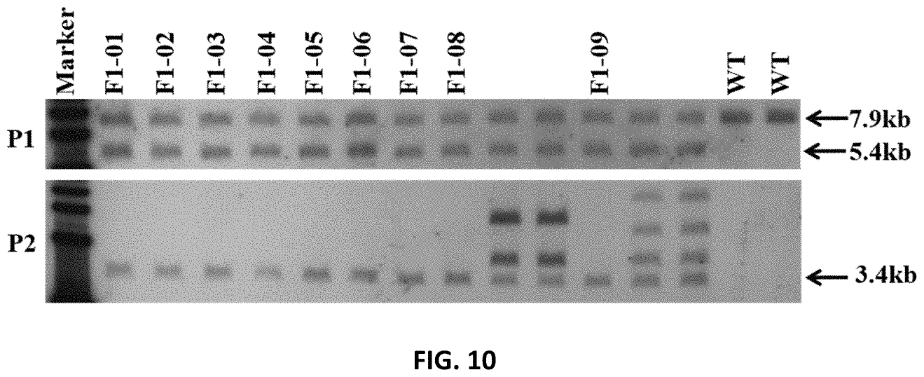

The detection result of Southern Blot is shown in FIG. 10. In view of the hybridization results by P1 and P2 probes, no random insertions were detected in the nine F1 generation mice numbered F1-01 to F1-09. The nine F1 generation mice were further verified by sequencing to be identified as positive heterozygotes and with no random insertions.

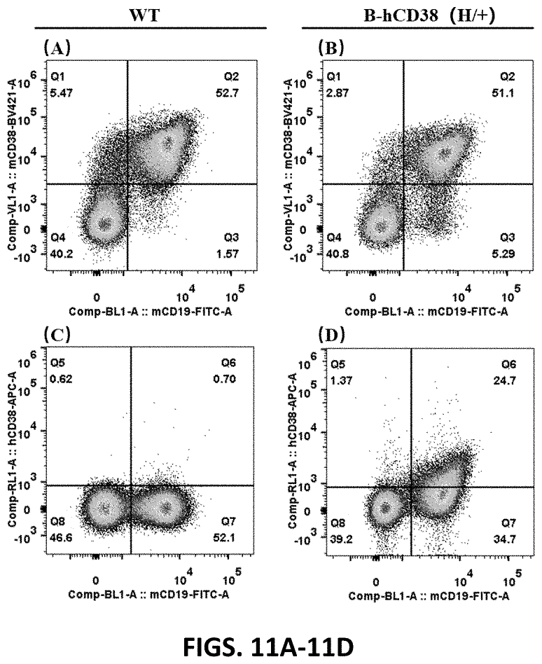

The expression of humanized CD38 protein in positive mice can be confirmed, e.g., by flow cytometry, etc. Specifically, flow cytometry was used to detect the expression of humanized CD38 protein in humanized CD38 gene heterozygous mice and homozygous mice. The detection method of heterozygous mice is as follows: one 6-week old wild-type C57BL/6 mouse and one CD38 gene humanized heterozygous mouse (produced by the methods described herein) were selected. Spleen cells and blood samples were collected after euthanasia. Anti-mouse CD38 antibody Brilliant Violet 421™ anti-mouse CD38 Antibody (mCD38-BV421-A) and anti-mouse CD19 antibody FITC anti-mouse CD19 Antibody (mCD19-FITC-A); alternatively, anti-human CD38 antibody APC mouse anti-human CD38 Antibody (hCD38-APC-A) and anti-mouse CD19 antibody FITC anti-mouse CD19 Antibody (mCD19-FITC-A) were used to stain the cells for flow cytometry analysis. The results for spleen samples are shown in FIGS. 11A-11D. Mouse CD38 protein can be detected in wild-type C57BL/6 mouse (FIG. 11A), but humanized CD38 protein cannot be detected in the wild-type C57BL/6 mouse (FIG. 11C). In CD38 humanized heterozygous mouse, both mouse CD38 protein (FIG. 11B) and humanized CD38 protein (FIG. 11D) can be detected.

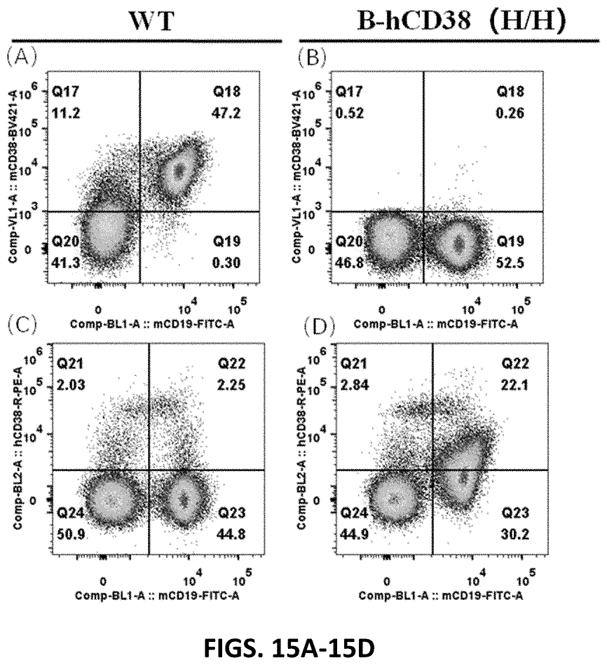

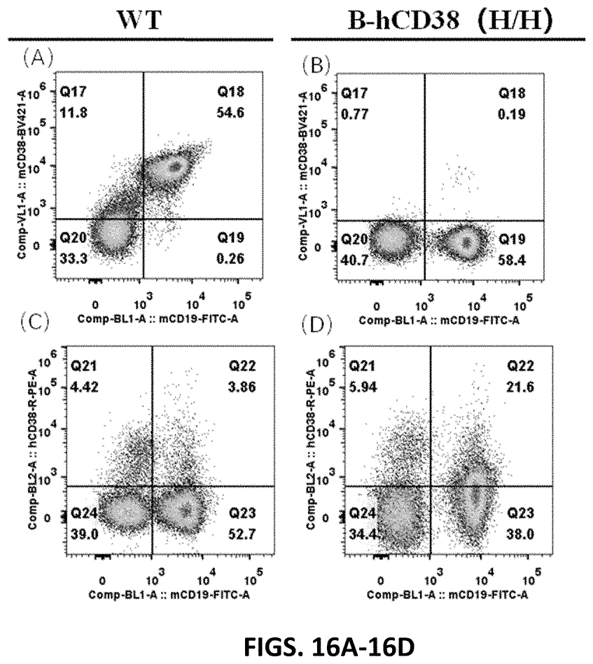

The detection method of homozygous mice is as follows: one 6-week old wild-type C57BL/6 mouse and one CD38 gene humanized homozygous mouse (produced by the methods described herein) were selected. Spleen cells and blood samples were collected after euthanasia. Anti-human CD45 antibody Brilliant Violet 510™ anti-mouse CD45 Antibody, anti-mouse CD19 antibody anti-mouse CD19 FITC Antibody (mCD19-FITC-A), and anti-mouse CD38 antibody Brilliant Violet 421™ anti-mouse CD38 Antibody (mCD38-BV421-A); alternatively, anti-human CD45 antibody Brilliant Violet 510™ anti-mouse CD45 Antibody, anti-mouse CD19 antibody anti-mouse CD19 FITC Antibody (mCD19-FITC-A), and anti-human CD38 antibody Human CD38 PE-conjugated Antibody (hCD38-PE-A) were used to stain the cells for flow cytometry analysis. The results for spleen cells and blood samples are shown in FIGS. 15A-15D and FIGS. 16A-16D, respectively. Mouse CD38 protein can be detected in wild-type C57BL/6 mouse (FIG. 15A and FIG. 16A), but humanized CD38 protein cannot be detected in the wild-type C57BL/6 mouse (FIG. 15C and FIG. 16C). In CD38 humanized homozygous mouse, mouse CD38 protein cannot be detected (FIG. 15B and FIG. 16B), but humanized CD38 protein can be detected (FIG. 15D and FIG. 16D).

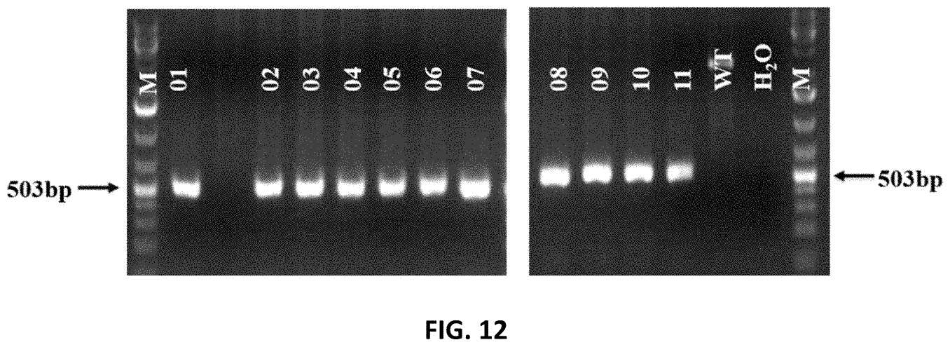

In addition, due to the double-strand break of genomic DNA caused by Cas9 cleavage, insertion/deletion mutations can be randomly generated through chromosome homologous recombination repair, which may result in knockout mice with loss of CD38 protein function. A pair of primers were designed to detect knockout mice. Wild-type mice should have no PCR bands. Knockout mice should have one PCR band, and the length of the PCR product should be about 503 bp. As shown in FIG. 12, 11 mice numbered 01 to 11 were identified as CD38 gene knockout mice. The primers are located on the upstream of the 5′ end targeting site and downstream of the 3′ end targeting site, with sequences shown as follows:

SEQ ID NO: 48:
5′-ACCATGTATGTGCAGTGACTGTGGA-3′
SEQ ID NO: 49:
5′-CACAGATAACAATCCGTTCACTAT-3′

### Example 2: Generation of Double- or Multi-Gene Humanized Mice

The humanized CD38 mouse prepared by the methods described herein can also be used to prepare a double- or multi-gene humanized mouse model. For example, in Example 1, the embryonic stem cells used for blastocyst microinjection can be selected from mice containing CD31, CD3, CD28, BCMA, PD-1, PD-L1, IL15R, A2aR, or other genetic modifications. Alternatively, the embryonic stem cells of CD38 gene humanized or knockout mice can be selected for gene editing, to obtain a double-gene or multi-gene humanized mouse model comprising humanized CD38 and other genetic modifications. In addition, it is also possible to breed the homozygous or heterozygous CD38 transgenic mice obtained by the methods described herein with other genetically modified homozygous or heterozygous mice, and the offspring can be screened. According to Mendel's law, it is possible to generate double-gene or multi-gene modified heterozygous mice comprising humanized CD38 gene and other genetic modifications. Then the heterozygous mice can be bred with each other to obtain homozygous double-gene or multi-gene humanized mice. These double-gene or multi-gene modified mice can be used to verify the in vivo efficacy of human CD38 and other gene regulators.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric CD38.

2. The animal of claim 1, wherein the sequence encoding the human or chimeric CD38 is operably linked to an endogenous regulatory element at the endogenous CD38 gene locus in the at least one chromosome.

3. The animal of claim 1 or 2, wherein the sequence encoding a human or chimeric CD38 comprises a sequence encoding an amino acid sequence that is at least 70%, 75%, 80%, 85%, 90%, 95%, 99%, or 100% identical to human CD38 (NP_001766.2 (SEQ ID NO: 4)).

4. The animal of claim 1 or 2, wherein the sequence encoding a human or chimeric CD38 comprises a sequence encoding an amino acid sequence that is at least 70%, 75%, 80%, 85%, 90%, 95%, 99%, or 100% identical to SEQ ID NO: 13 or 58.

5. The animal of claim 1 or 2, wherein the sequence encoding a human or chimeric CD38 comprises a sequence that is at least 70%, 75%, 80%, 85%, 90%, 95%, 99%, or 100% identical to amino acids 79-300 of SEQ ID NO: 4.

6. The animal of claim 1 or 2, wherein the sequence encoding a human or chimeric CD38 comprises a sequence that is at least 70%, 75%, 80%, 85%, 90%, 95%, 99%, or 100% identical to amino acids 43-300 of SEQ ID NO: 4.

7. The animal of any one of claims 1-6, wherein the animal is a mammal, e.g., a monkey, a rodent, a rat, or a mouse.

8. The animal of any one of claims 1-7, wherein the animal is a mouse.

9. The animal of any one of claims 1-8, wherein the animal does not express endogenous CD38.

10. The animal of any one of claims 1-9, wherein the animal has one or more cells expressing human or chimeric CD38.

11. The animal of any one of claims 1-10, wherein the animal has one or more cells expressing human or chimeric CD38, and human CD31 can bind to the expressed human or chimeric CD38.

12. The animal of any one of claims 1-10, wherein the animal has one or more cells expressing human or chimeric CD38, and endogenous CD31 can bind to the expressed human or chimeric CD38.

13. A genetically-modified, non-human animal, wherein the genome of the animal comprises a replacement of a sequence encoding a region of endogenous CD38 with a sequence encoding all or a portion of the extracellular region of human CD38, thereby generating a chimeric CD38 gene at an endogenous CD38 gene locus.

14. The animal of claim 13, wherein the animal is a mouse, and the sequence encoding a region of endogenous CD38 is within exon 1 of the endogenous mouse CD38 gene.

15. The animal of claim 14, wherein the sequence encoding all or a portion of the extracellular region of human CD38 further comprises Woodchuck Hepatitis Virus (WHP) Posttranscriptional Regulatory Element (WPRE) and/or polyA (polyadenylation) signal sequence.

16. The animal of claim 13, wherein the animal is a mouse, and exon 1, exon 2, exon 3, exon 4, exon 5, exon 6, exon 7, and/or exon 8, or a part thereof, of the endogenous mouse CD38 gene is replaced.

17. The animal of claim 16, wherein a sequence starting within exon 2 and ending within exon 3 of the endogenous mouse CD38 gene is replaced.

18. The animal of claim 17, wherein the sequence encoding all or a portion of the extracellular region of human CD38 further comprises mouse CD38 3′UTR and/or LoxP STOP sequence.

19. The animal of any one of claims 13-18, wherein the chimeric CD38 gene is operably linked to an endogenous regulatory element at the endogenous CD38 locus, and one or more cells of the animal expresses a chimeric CD38 that is encoded by the chimeric CD38 gene.

20. The animal of any one of claims 13-19, wherein the animal does not express endogenous CD38.

21. The animal of any one of claims 13-20, wherein the animal has one or more cells expressing a chimeric CD38 having a cytoplasmic region, a transmembrane region, and an extracellular region, wherein the extracellular region comprises a sequence that is at least 50%, 60%, 70%, 80%, 90%, 95%, 99%, or 100% identical to the extracellular region of human CD38.

22. The animal of claim 21, wherein the extracellular region of the chimeric CD38 has a sequence that has at least 10, 20, 30, 40, 50, 60, 70, 80, 90, 100, 110, 120, 130, 140, 150, 160, 170, 180, 190, 200, 210, 220, 230, 240, or 250 contiguous amino acids that are identical to a contiguous sequence present in the extracellular region of human CD38.

23. The animal of any one of claims 13-22, wherein the animal is heterozygous with respect to the chimeric CD38 gene.

24. The animal of any one of claims 13-22, wherein the animal is homozygous with respect to the chimeric CD38 gene.

25. A method for making a genetically-modified, non-human animal, comprising:
replacing in at least one cell of the animal, at an endogenous CD38 gene locus, a sequence encoding a region of an endogenous CD38 with a sequence encoding all or a portion of the extracellular region of human CD38.

26. The method of claim 25, wherein the sequence encoding all or a portion of the extracellular region of CD38 comprises exon 1, exon 2, exon 3, exon 4, exon 5, exon 6, exon 7, and/or exon 8, or a part thereof, of a human CD38 gene.

27. The method of claim 25 or 26, wherein exon 1, exon 2, exon 3, exon 4, exon 5, exon 6, exon 7, and/or exon 8, or a part thereof, of the endogenous CD38 gene is replaced.

28. The method of any one of claims 25-27, wherein the sequence encoding all or a portion of the extracellular region of human CD38 encodes amino acids 43-300 of SEQ ID NO: 4.

29. The method of claim 28, wherein a sequence within exon 1 of the endogenous CD38 gene is replaced.

30. The method of any one of claims 25-27, wherein the sequence encoding all or a portion of the extracellular region of human CD38 encodes amino acids 79-300 of SEQ ID NO: 4.

31. The method of claim 30, wherein a sequence starting within exon 2 and ending within exon 3 of the endogenous CD38 gene is replaced.

32. The method of any one of claims 25-31, wherein the animal is a rodent, a rat, or a mouse.

33. A non-human animal comprising at least one cell comprising a nucleotide sequence encoding a chimeric CD38 polypeptide, wherein the chimeric CD38 polypeptide comprises at least 50 contiguous amino acid residues that are identical to the corresponding contiguous amino acid sequence of a human CD38, wherein the animal expresses the chimeric CD38 polypeptide.

34. The animal of claim 33, wherein the chimeric CD38 polypeptide has at least 50 contiguous amino acid residues that are identical to the corresponding contiguous amino acid sequence of a human CD38 extracellular region.

35. The animal of claim 33, wherein the chimeric CD38 polypeptide has at least 100 contiguous amino acid residues that are identical to the corresponding contiguous amino acid sequence of a human CD38 extracellular region.

36. The animal of claim 33, wherein the chimeric CD38 polypeptide has at least 200 contiguous amino acid residues that are identical to the corresponding contiguous amino acid sequence of a human CD38 extracellular region.

37. The animal of any one of claims 33-36, wherein the chimeric CD38 polypeptide comprises a sequence that is at least 80%, 85%, 90%, 95%, or 99% identical to amino acids 43-300 of SEQ ID NO: 4.

38. The animal of any one of claims 33-36, wherein the chimeric CD38 polypeptide comprises a sequence that is at least 80%, 85%, 90%, 95%, or 99% identical to amino acids 79-300 of SEQ ID NO: 4.

39. The animal of any one of claims 33-38, wherein the nucleotide sequence is operably linked to an endogenous CD38 regulatory element of the animal.

40. The animal of any one of claims 33-39, wherein the chimeric CD38 polypeptide comprises an endogenous CD38 cytoplasmic region and/or an endogenous CD38 transmembrane region.

41. The animal of any one of claims 33-40, wherein the nucleotide sequence is integrated to an endogenous CD38 gene locus of the animal.

42. The animal of any one of claims 33-41, wherein the chimeric CD38 polypeptide has at least one mouse CD38 activity and/or at least one human CD38 activity.

43. A method of making a genetically-modified mouse cell that expresses a chimeric CD38, the method comprising:
replacing at an endogenous mouse CD38 gene locus, a nucleotide sequence encoding a region of mouse CD38 with a nucleotide sequence encoding all or a portion of the extracellular region of human CD38, thereby generating a genetically-modified mouse cell that includes a nucleotide sequence that encodes the chimeric CD38, wherein the mouse cell expresses the chimeric CD38.

44. The method of claim 43, wherein the chimeric CD38 comprises a cytoplasmic and/or a transmembrane region of mouse CD38; and all or a portion of the extracellular region of human CD38.

45. The method of claim 43 or 44, wherein the nucleotide sequence encoding the chimeric CD38 is operably linked to an endogenous CD38 regulatory region, e.g., promoter.

46. The animal of any one of claims 1-24 and 33-42, wherein the animal further comprises a sequence encoding an additional human or chimeric protein.

47. The animal of claim 46, wherein the additional human or chimeric protein is CD31, CD3, B-cell maturation antigen (BCMA), interleukin-15 receptor (IL15R), adenosine A2a receptor (A2aR), programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα), or TNF Receptor Superfamily Member 4 (OX40).

48. The method of any one of claims 25-32 and 43-45, wherein the animal or mouse further comprises a sequence encoding an additional human or chimeric protein.

49. The method of claim 48, wherein the additional human or chimeric protein is CD31, CD3, BCMA, IL15R, A2aR, PD-1, CTLA-4, LAG-3, BTLA, PD-L1, CD27, CD28, CD47, CD137, CD154, TIGIT, TIM-3, GITR, SIRPα, or OX40.

50. A method of determining effectiveness of an anti-CD38 antibody for treating cancer, comprising:
administering the anti-CD38 antibody to the animal of any one of claims 1-24 and 33-42, wherein the animal has a cancer; and
determining the inhibitory effects of the anti-CD38 antibody to the cancer.

51. The method of claim 50, wherein the cancer comprises one or more cells that express CD38.

52. The method of claim 50 or 51, wherein the cancer comprises one or more cancer cells that are injected into the animal.

53. The method of any one of claims 50-52, wherein determining the inhibitory effects of the anti-CD38 antibody to the cancer involves measuring the tumor volume in the animal.

54. The method of any one of claims 50-53, wherein the animal has multiple myeloma, acute lymphoblastic leukemia, acute myeloid leukemia, hematological malignancy, solid tumor, and/or non-Hodgkin's lymphoma.

55. A method of determining effectiveness of an anti-CD38 antibody and an additional therapeutic agent for treating cancer, comprising
administering the anti-CD38 antibody and the additional therapeutic agent to the animal of any one of claims 1-24 and 33-42, wherein the animal has a cancer; and
determining the inhibitory effects on the cancer.

56. The method of claim 55, wherein the animal further comprises a sequence encoding a human or chimeric programmed cell death protein 1 (PD-1).

57. The method of claim 55 or 56, wherein the animal further comprises a sequence encoding a human or chimeric programmed death-ligand 1 (PD-L1).

58. The method of any one of claims 55-57, wherein the additional therapeutic agent is an anti-PD-1 antibody or an anti-PD-L1 antibody.

59. The method of any one of claims 55-58, wherein the cancer comprises one or more cancer cells that express CD38, PD-L1 or PD-L2.

60. The method of any one of claims 55-59, wherein the cancer is caused by injection of one or more cancer cells into the animal.

61. The method of any one of claims 55-60, wherein determining the inhibitory effects of the treatment involves measuring the tumor volume in the animal.

62. The method of any one of claims 55-61, wherein the animal has melanoma, pancreatic carcinoma, mesothelioma, hematological malignancies (e.g., Non-Hodgkin's lymphoma, lymphoma, chronic lymphocytic leukemia), and/or solid tumors.

63. A method of determining effectiveness of an anti-CD38 antibody for treating an allergic disorder, comprising:
administering the anti-CD38 antibody to the animal of any one of claims 1-24 and 33-42, wherein the animal has the allergic disorder; and
determining the inhibitory effects of the anti-CD38 antibody.

64. The method of claim 63, wherein the allergic disorder is asthma.

65. A method of determining effectiveness of an anti-CD38 antibody for reducing inflammation, comprising:
administering the anti-CD38 antibody to the animal of any one of claims 1-24 and 33-42, wherein the animal has the inflammation; and
determining the inhibitory effects of the anti-CD38 antibody.

66. A method of determining effectiveness of an anti-CD38 antibody for treating autoimmune disorder, comprising:
administering the anti-CD38 antibody to the animal of any one of claims 1-24 and 33-42, wherein the animal has the autoimmune disorder; and
determining the inhibitory effects of the anti-CD38 antibody.

67. The method of claim 66, wherein the autoimmune disorder is rheumatoid arthritis, inflammatory bowel disease, systemic lupus erythematosus, or multiple sclerosis.

68. A method of determining toxicity of an anti-CD38 antibody, the method comprising administering the anti-CD38 antibody to the animal of any one of claims 1-24 and 33-42; and
determining weight change of the animal.

69. The method of claim 68, the method further comprising performing a blood test (e.g., determining red blood cell count).

70. A protein comprising an amino acid sequence, wherein the amino acid sequence is one of the following:
(a) an amino acid sequence set forth in SEQ ID NO: 13 or 58;
(b) an amino acid sequence that is at least 90% identical to SEQ ID NO: 13 or 58;
(c) an amino acid sequence that is at least 91%, 92%, 93%, 94%, 95%, 96%, 97%, 98%, or 99% identical to SEQ ID NO: 13 or 58;
(d) an amino acid sequence that is different from the amino acid sequence set forth in SEQ ID NO: 13 or 58 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 13 or 58.

71. A nucleic acid comprising a nucleotide sequence, wherein the nucleotide sequence is one of the following:
(a) a sequence that encodes the protein of claim 70;
(b) SEQ ID NO: 8, 9, 12, 50, 51, 54, 55, or 57;
(c) a sequence that is at least 90% identical to SEQ ID NO: 8, 9, 12, 50, 51, 54, 55, or 57; and
(d) a sequence that is at least 91%, 92%, 93%, 94%, 95%, 96%, 97%, 98%, or 99% identical to SEQ ID NO: 8, 9, 12, 50, 51, 54, 55, or 57.

72. A cell comprising the protein of claim 70 and/or the nucleic acid of claim 71.

73. An animal comprising the protein of claim 70 and/or the nucleic acid of claim 71.

74. A method for making a genetically-modified, non-human animal, comprising:
replacing in at least one cell of the animal, at an endogenous locus that encodes an endogenous protein, a sequence encoding a region of the endogenous protein with a sequence encoding all or a portion of the extracellular region of the corresponding protein in human;
wherein the endogenous protein comprises from N-terminus to C-terminus: a cytoplasmic region, a transmembrane region, and an extracellular region.

75. The method of claim 74, wherein the endogenous protein is a type II transmembrane protein.

76. The method of claim 74 or 75, wherein the endogenous protein is endogenous CD38 and the corresponding protein in human is human CD38.

77. A method for making a genetically-modified, non-human animal, comprising:
inserting in at least one cell of the animal, at an endogenous locus that encodes an endogenous protein, a sequence encoding all or a portion of the extracellular region of the corresponding protein in human; wherein the endogenous protein comprises from N-terminus to C-terminus: a cytoplasmic region, a transmembrane region, and an extracellular region.

78. The method of claim 77, wherein the method further comprises deleting a sequence encoding a region of the endogenous protein.

79. The method of claim 77 or 78, wherein the endogenous protein is a type II transmembrane protein.

80. The method of any one of claims 77-79, wherein the endogenous protein is endogenous CD38 and the corresponding protein in human is human CD38.

81. A chimeric CD38 protein, wherein the chimeric CD38 protein comprises a humanized CD38 extracellular region.

82. The chimeric CD38 protein of claim 81, wherein the chimeric CD38 protein comprises a rodent cytoplasmic region and/or a rodent transmembrane region.

83. The chimeric CD38 protein of claim 81, wherein the chimeric CD38 protein comprises a rodent cytoplasmic region and/or a chimeric transmembrane region.


SEQUENCE LISTING (59 sequences)

SEQ ID NO: 1 - DNA, 3013 bp, Mus musculus
gcaggctctgacccagtcagccctgtgctctcttcctgcctagcctgggccagtcttcgggagcccaatggctaactatgaatttagccaggtgtctggggacagacctggctgccgcctctctaggaaagcccagatcggtctcggagtgggtctcctggtcctgatcgccttggtagtagggatcgtggtcatacttctgaggccgcgctcactcctggtgtggactggagagcctaccacgaagcacttttctgacatcttcctgggacgctgcctcatctacactcagatcctccggccggagatgagagatcagaactgccaggagatactgagtacattcaaaggagcatttgtttccaagaacccttgcaacatcacaagagaagactacgccccacttgttaaattggtcactcaaaccataccatgtaacaagactctcttttggagcaaatccaaacacctggcccatcaatatacttggatccagggaaagatgttcaccctggaggacaccctgctgggctacattgctgatgatctcaggtggtgtggagaccctagtacttctgatatgaactatgtctcttgcccacattggagtgaaaactgtcccaacaaccctattactgtgttctggaaagtgatttcccaaaagtttgcagaagatgcctgtggtgtggtccaagtgatgctcaatgggtccctccgtgagccattttacaaaaacagcacctttggaagtgtggaagtctttagtttggacccaaataaggttcataaactacaggcctgggtgatgcacgacatcgaaggagcttccagtaacgcatgttcaagctcctccttaaatgagctgaagatgattgtgcagaaaaggaatatgatatttgcctgcgtggataactacaggcctgccaggtttcttcagtgtgtgaagaaccctgagcacccatcgtgtagacttaatacgtgaaggatctggatcttagatcacctgtagcctggactgagatgaaggggctcagaagcaacactggtggaaagctgaaactgtcagggagaagcctctactacagtgttaacaccagagatggaagaacttcccaattctctgtgtactaccaacattcaagaaaaattactccataaaccagagttaaacttctatattgttatattagtctaactttctcatgtggtgcttctgtattgtttatatattgcttacatccttttattcctcttttaatgatctctcttttctctctctctctctctctctctctctctcaatgaggctgagaatccaacctgagaactttcatacatggtggataagcctatataccactgagctaaatcctcagcacagctgataacatcatttttgctgaaaaatggccaatcaaacttcccattagacaaagaaagtcaaatgtcaagtatatcgaaatgaatgacccttttttttttttatgttttttcattcttcccacagatattcacatggtaaacctgaggtcatagggtcattatagggaaggtgctgtgtgggaactacccacgtgccctgtgctttaatctttaactcaacacatccctgataactttgagcattcttttcttttcttttctttccttttcttttcttttcttttctttttcttttttttaaacagaagaaaatggagtttagagagataattgattttcctaaagtcacattcttaagatcatggttacatctagaattaaatcagttctgtcaaagtccacaacatatgatagtttttttcttttccgagcttttattgccaaattggtagtagcctgtgttgtcgtctagcaaatgctattgcattagattgaatataatagaatctgaaatgtgatctttatggaagatcacaaaactaattacttgcaccctctttctcttccacttcattcttggtgccttttataggcagtcaagagttaacatatatttgttgctaatattggcaatttatgttttctatcctttttttcttagatcagtcttgctcagaatcactggttttgttatgttgttgttgttgctattgttattgttcttgtctttttcaaagctcaaatctttaactttcctcatttatttatttaattgagctttagtctttatttttagaaacatagaaatttcttctagctatctggaatatcttcagttttttgaggcaaatgcttagaccattgatatttcagtctgttttctacacatgtactttaggattctaggtttctccctgagccctgctttcgatgtaacactgaatttctgtatgtctttactggttagttactttgatagtttgtatatgcttgacccagtgagtggcaggattagaaggtatggccttgctggaataggtgtgccactgtgggtgtagtcttaagacccttaccctagctgcctggaggccactattccactaacagccttcaaatgaaaatataaaactctcagctctgcctgtgccatgcctgcctggatgctgccatgctcccaccttgatgataatggactgaacctctgaacctgtaagccagccccaatttgttgtccttataaaagacttgctttggtcatggtatctgttcacagcagaaagaacctaactaagacagttaccattcagttcaaaataattcttgattttattgttatttagacatgtgatatttacttttcaacatctggagaattgtttaggttttttttgtgtgtgtgtctttagtaggtattaataaactaaattgagacaactccaaagtatttgcttggtttattgaaaatagcataaacagagccaggtgcttctgtattcatgttttgacaccagtttgtgattgttgaacaatatatcttatttctaaacttttaaaaagctacttaaagacattaacttgctcccttacaatatgttgatggttcagtccattaaatgcttactttgtaagt

SEQ ID NO: 2 - protein, 304 aa, Mus musculus
MANYEFSQVSGDRPGCRLSRKAQIGLGVGLLVLIALVVGIVVILLRPRSLLVWTGEPTTKHFSDIFLGRCLIYTQILRPEMRDQNCQEILSTFKGAFVSKNPCNITREDYAPLVKLVTQTIPCNKTLFWSKSKHLAHQYTWIQGKMFTLEDTLLGYIADDLRWCGDPSTSDMNYVSCPHWSENCPNNPITVFWKVISQKFAEDACGVVQVMLNGSLREPFYKNSTFGSVEVFSLDPNKVHKLQAWVMHDIEGASSNACSSSSLNELKMIVQKRNMIFACVDNYRPARFLQCVKNPEHPSCRLNT

SEQ ID NO: 3 - DNA, 5620 bp, Homo sapiens
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 4 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 5 - DNA, 1349 bp, Artificial Sequence
  LoxP STOP sequence
attaagggttccggatcctcggggacaccaaatatggcgatctcggccttttcgtttcttggagctgggacatgtttgccatcgatccatctaccaccagaacggccgttagatctgctgccaccgttgtttccaccgaagaaaccaccgttgccgtaaccaccacgacggttgttgctaaagaagctgccaccgccacggccaccgttgtagccgccgttgttgttattgtagttgctcatgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacatggtaagtaagcttgggctgcaggtcgagggaccta

SEQ ID NO: 6 - DNA, 3936 bp, Artificial Sequence
  5′ homologous arm
gttatacagtgaaatgttgttcagcctttaaaagggaggaaatttaatacatacgtggaggtttgaatgggaatgacccccacaggctcaaatatgtgaatttttggtttccagttgaactatttaggaatgactgggaagtgtggccttattggaggcgtgtccctgggagtgggttttaatgtttcaaaagtccatgccagactcagactcccccccacaaacacacccccaccacccgctccttgcagattagaatgtgcattcttagttactgctccagcgtgatgctctctgtcctctgagactctccctctgaaaccctaagcaagctcccaattaaaagctttcttctatcagctgctttggtcatgatgtaacctgatcatagcaggagcgaccctaattaagatgtaatatagctgggaacatcatgctaagtaaaattagccatttacaaagattccaggtttgtgagatgcctatcagattgaaggaaatagaaagaattgtggtagccagggactgggtaaaggaggggtggggtgttaatacggatgagttacaaacatcctgactatttacctggacacttaaacagagtcaagcagaaggtgcatctcagtggtagagcacttgcctagcatgtgtgatactgtatttagttcctagtactaaacaaaacaaaacaaaacaaaacaaaacaaaacaaaacaaaacaaaacaaaacaaaacaaaacaaaactacccagtactgttaggtgtcatagtcccaaaactcataaattctgttattataaccaaagtagagtttgcatcttgatgattgcaaactaaagacaagcacagctgagaaataagatcagagaaagagttgctgcaagcagcaggctcaagaagaaaagatagtagacacttttgaggcatgcttaggtaaaaattgtgaagcatatttttgcaggattacagtggtggatgagaacaattttgggggtgctcaccttgaatcataaagatggtaaccagtatcacttggcatgctcaactgtctttaaagaaagaaaagttttagctggaaaaaggaaaggatattttaaatgtcttactgtagaactttgcccctaaaagttagatcttaacattatttttaatttgttacaaggtaaaaaaaattcaaatacatagagaaatttaaaaagtattagaaaacaaaacaacaacaacaataacaacaataaacgaacaaaacaaaacattatttcctggagcagccaggaaaaagacacctaagtgcccagagctactcccctgagtgaatatgtgaagaacaggggaaagagggagaagagagagtacttagtggagagacagaagagaggagggtggagctgagcaaatcatgtactgtagagagaagtggaactggatagcagttagaaacaggcagacatgaggagccatggtgatgtctgagcccgggtgctaccaagggccgtgtctgcttctgtggcactgctccagccagagtctgtgctgatgtctgtggccagagttaacaccaaagaccatgaggatgtctctggcatgggctgccacttaaggccctgttgatgtccaagcactatacaaggctggctctgcctcttgcctaggcaccatgggagagctggctctggtgctggggatgctgaagagctggtcccagtaggatgagtgtgggagagctggccctgtgtccctcaccaaatgcagcactcagatccccacatctcttcccctgggcagcaccgtataactggtcgtggtaatgagagcaagggtgagtggccagagggcatgtgagcaggagagctggacctgcccctggcttggttaaagtaggagagttggtcccagtggtacatgtatgggagagctggtgagctgaccaactcagtttccacctaggcccagatccaggggtctgagttggcccaccagaacatccaccccatctatgaactgctggagcaaatgaagaggctggtcctgcagatcccgagctgtgggcactccatgacacagggtgacaacaggataactgagaagagttccagtgagggtccaatattgatgatgtgacaaaagctagaggctttgggaaagaccaatcaatgacttattgcaatgaacatttgcaaggaaagatgtgtggacaaatgcgtatactgtggtatgtgccatgacaggcggcaacttccacagcaagatttttatttaattcattttattttttgttcacttttgggtagggggaggctgccggactgaagggtaaaatgggtgggtttggaatgcatggtgtgaaattcacaaaggaccaataatttttttttaattttaaaaactgtacttataatgtatgcaagatgtttttgttattctctgcaaattaccaactcttccaagaatcaagtcttccaagtcatgttcagcagtactactccagtctcctctcctgagcagacagccctcctccagtgggtatcttgaatctttattacttcttgtactggaatataagacctaagagaagcatagaaataatttgtgaaggacgtgagtttcagcacttaaataagagtttgctgatgagaaggtctgctgtgtggcagggtctcagggcaggataactgctctttcccaccacgcttccagaagtggaaaatgttctctccttcttccatcttcttctgtttcaagtcctgcagatgctccaaaggaaagacgtagagatttttatgacggttgggacttagaacagagagttcttgagaaggccactgcattaattaccagaaagaaaacataaaccctggaagccgggaagccgccaaacgtctattttctcaattgcctgtgatggtactctataaattttgattgacactaaagtacatgatgtcatcacttttcctttttatgtatgtaaacggtacggaaggagagggccctaatggtttcctggcatcgttttcaaaggcaggtgctgatgaacttaagcttctcagtctagactctggaaagaaaccccatcaaacctttcacattttcatttgtatcccctgattctcaacccgaactagggtcagaactgcctggggtgggggtgggggtggggtggaagtgggaggtatgaatgccactgatgatcccgcccaacattaccaagcccacctgcagtaaccctacctacttaaagttttagtctgacacagagtaattggacatatttatgagtacaatgtggcatttggcctatgtatacactgtataaagaacagtgacagacagctctatcgcctcaggtgctcatgacttctctgtgttgggaacattcggtcctctctacttgctgttttgaaacattgcccttactgggggtactaaagaaccttaaggactttttttatacaaggtgtagtatgagacagaaaacgctggtgtcttcctattttcttcttaatgttattagatgcttgggtcattttaatttcacagagcctcagcctcaggtaattttcatctcacagggacattttacacagctaataggatggtgtcaagaactgactgcccctgggtatatgattccagatgaagggtatttcttgttgctatctatgttccaaaaatattttttaaactttttcaatgtctttctttttttcttttatttattttttttatatgttcctaagattgatatttattttacatgtagagttgttttgtctgtagatatgttcatgcaccatgtatgtgcagtgactgtggaagccagatggaggtgttagctctggaaactgaacacactgttgggagccaccgtgagccatctctccagactcttcaaaaatatttttaaaatctgttttccatctttcattttataga

SEQ ID NO: 7 - DNA, 5392 bp, Artificial Sequence
  3′ homologous arm
taagacatccccaaatccagtctctccctcactttccctctctacatccttcctgttctccccttttcctgcctcttccactttcctcttttccctcctccctctccactttcttctttctcttctgcacttttggcaagaaatctcctccttaacctttatatatgtatttacacacacacacacacacacacacacacacacacacgttatatattcttgaacataactctaagtcatttttttaaaaaaatttaaattaaaattatatatatttttatctgtacagagtaatattggatataaatagacatagtgaacggattgttatctgtgagctatttaacatggacaactttgggtaccatcttaaggtgatctctttctttccccttcatagccagtgatgatctatttgcctagagagccatctgtgcatctcccctcacactgccttttagcaccatcacaccctctccatcctgctgggaatgctataaggttttactggtcactctttaccctcctaggtcagtctccatctgacaccatagtttttctcagagtgaaaatgggatgatgtcgatctccctctagaacctaacaatctctcttttgactctaggctaatggtctaaagacctgacctctgtgtttcagatgcctcaccttctcattcccttctccatcctcttttctgatcaaagctcatcatggctctttgcctttgcaccgtgccataggcacaaggtgcccttctctttagagtctgtctgaagacagtgattcttcttcaaagtaaatttcaacgtgaatctgtctctgattatttgacttcactacatcacatgtcctatgaggcagggctcccacctgattcaaccctctatctccagaacctaaccttcagtaaatgtttatatatatatgtgtgtgtgtgtgtgtgtgtgtttatatatatatgtatatatatatgtatatatatatatatatgcaaataaccttgagaacagtgtcatagaaaccatgcttctactctatactttgcaccacactaagggctaaacctgttttggtagggttgactatagaaagcctgagatcacctgagcccccaaatttatggaggttgaaaaggaaactctccataaaagagaattgccaggaatcagaggactatctgggggaaagtgtcttcttcccgaaaaggcctggccttctccctaagtgttcctggactccaggggagtgagaggggccggtgtctttagggtgcacttttgttgccatttcccagaatggtgagtcaaaacccatcacagtgttcctgtctgggatctggacggggaggtaaagagttggaaggaaatcaggggacacttacttaaattgggttgagctgtgtccattctccagcctctgtcttggcacagatgctgttggaaagttctcctatggggattggacagccaagaacggagcccaaaggtacatttcctttccatttaactgttttctataaacgatgtgttttagatatgaactatgtctcttgcccacattggagtgaaaactgtcccaacaaccctattactgtgttctggaaagtgatttcccaaaaggtaagtcacaaatagttaaattctagagttttcactgaagatactgttcagtccatgactggttgacttctggccctttgggtggggggtacagatgattttaggtgtaaaagtatgaaaggaaatcatgccagaagatttttctacacaacttcagaacacccaggaaggcttgggagctcaggggaagaaacctaacccagtgaggaggtaacactcactggctgtgtctaccaggatgctaggcttggcttagtcctgggacccaggtattgacttgatttctggaacagtcttaataattggcatttgtagaccacttgatgtgtgtccaactctttgcaggagttaagaactctagcctcacaacagccatatgaaacaccatcaatatcacgactacttgtaaaaatcttttttaaaatttgtttacctttatttttatatgaattggtgtttttgcctgcatgtctgtctatgtgagagtgtcagatcttggagttacaaactgccatatgggtattaggatttgaacccaggttctctggtagatctgtcagtgcctttaactgctgcactatatttccaacccccaaaaccaattttcttagacattctgatcagtttccttatttcatcttcaccaaatggaaattaacacatacctagtctagatagctattggtgcattcaaataaaatatcactagcttgttaagtaatggaacccacctacacaacaaatgtttattgaagacttactgcatgttttaaaaactcaaagacgctgtatatttttgtttgctcgcttttacagagaatgttttccatttgtgttatcactttgggatatgttttacagccacatcatctctgaaaccccctacagcagttcatgttttatataaactaaatgcttccttgtatcacatctcagccttggtaccaaggctaagatgctgagggtgattggaggcaccttggcttctgagacactgtgcacctgtaccttgttctaggatctctttgaggaagacaaacaaactagtccctctttttgacaataaggatggttcttcttacaggtttgaattcccagatcaccttgttgctaagttgtgtgaccttgaggccaacggtgccaatgcttactttgtgtcagaggacttgtgagaattctcctttatagggttcaggactcttctgggtcagtgcttaatgattaatcataaggagtaggacttggtccctctgtggttacacaagcaccactggacttcataataaagctctgaatctatctgtagtgtagaagtcacccgcagagtaaagtgcaggttccccaacaaagtacaaggccatctatgaactgaatattcaatatcgactgcaactttccccacaaactactgttctcctaaaccaagggccccttccagtcacagcttcaccagtggagtcctgaccgctctttgggacgtgggttctgtttgctatacagcactttcttggtctttcctagtgtccagtgcagagtatcttcttttctcctgtcatccttttcatccctggaattttactgagtaaaacatacctgtgtcatgcactaggtataaagcactgaaagttctagaagcacataggaatccaagctctttgttctcacaaagctcttcgtactgacggctaaaactgttcaacccgccataacaatgaaatgttgttaagtaatgtgcacagatgtcgtggaaggagacccacgtcccagaggtctgtctccaaaggaagctgaacagtactccgttcacatggtgacctccagtgcttagcagacctcccagctgaaagttggttgcatgaagaaataaaactccttgttcaaccatgttcttttattcaagcaccacactccctccttctttatgcctttctagagcctcaggatcactgcctcttacctgggcttccctttctacagtgttcccctaaattgtccaccttacttctgctaaataagatgtagcatctacagaacatgataccaagtgagtgctttcattcatgcaaattgccatggctgcattaagaattcatacaaagtaaattcttcctgtggcttagactattggatagattcaccattcttgtactagaactagggaggctattctctctccctctctagttagtactaagtactctttggatggaattgctgaagaataatgatgaacttccttttcttcctcctcttcttcctcttcctcctctttctcctcctcctcctcttcctcttcttccttctcttcttggttttggtctttgacagggtttctctgtatagctttggctgtcctggacatcactctgtagaccaggctactccaaactagcagagatccaaccacctgcctttgcctcccaagtgctgcaatcaaaggcatgtgtcaccactcagtctggctgacggacttcttttttaagtttgcagaagatgcctgtggtgtggtccaagtgatgctcaatgggtccctccgtgagccattttacaaaaacaggtactcatttcctttctttgtgcaagggttaattatcacagttaccatagtctcctgatggacatggcattcctagaaaggatgtactttccatgtttcatatcagtcctgaagtgctgagcttttaaaagggaaagcttatggattcgctcctttctgggtcaatgtttggtctggttcatctaagatcctccaaagttcctaaaggccattgcaggttcccagtcattgcccttgactttttgaggtctcacataattttcttagcttccttccccctgcattctatgctctcatttcatcgatactgtctaccctgcatctacccgtctccctgttagcatcctcaccatggctctctcctgtgtgaagagcaggaatcccacgtcattggaacatcagaaaccatgcagggctgtgatcctcctcctcctcttttggaagtgttgccagcaaagtcctgtagtgatataatggtgaagacaccagaggtccttatacatactatatgtgaccttgtgcaggcatctagcctctaggttgaggatgcatagaaagaaactgagataaagtgtgaacagtgctgcactccatgcctattgaaaattctctagtacatattagcttttttttttctttttcatttaatcctcagggctgagttggtccacactcaagccttccttctctggcctccttctcgccttagtttctatcatcatcatggtcttgttctctaatctttctctgatgagaatgcttctaaaaatgagagcacatattaattttcttcaggcactgtagataccctcaattaattgttcctttcttctgctttagtatttagtattataaagccacatttaccttttttccttcttctggacctttttttgggggtggggtggggggggctttgggtgaaagtgctggtgttcaaaatctcaccctctgatgttggagacccaagtccttgcctcaggatgggtagctgtccacactaacaagaaaggatctagaaagatgttgttgaatggacagtctctgtttctaactagctctctggtaatgctactctttgagcagcaagtatttttttaatgtatcagtcagcatcgatgtacagttaaaactcttatcttgaaaatctcataaccaagagtcatgttgaaagaggaacagtgggaagcaagggaggttctgttcaatcgtg

SEQ ID NO: 8 - DNA, 669 bp, Artificial Sequence
  the nucleotide sequence 322-990 of NCBI accession number NM_001775.4
catgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctga

SEQ ID NO: 9 - DNA, 80 bp, Artificial Sequence
  connection between the upstream of the human CD38 gene sequence and the mouse CD38 gene locus
aatatttttaaaatctgttttccatctttcattttatagacatgtagactgccaaagtgtatgggatgctttcaagggtg

SEQ ID NO: 10 - DNA, 80 bp, Artificial Sequence
  connection between the upstream of the Neo cassette and the downstream of the loxP STOP sequence
gacatggtaagtaagcttgggctgcaggtcgagggacctagaattccgaagttcctattctctagaaagtataggaactt

SEQ ID NO: 11 - DNA, 80 bp, Artificial Sequence
  connection between the downstream of the Neo cassette and the mouse sequence
gtataggaacttcatcagtcaggtacataatggtggatcctaagacatccccaaatccagtctctccctcactttccctc

SEQ ID NO: 12 - DNA, 3013 bp, Artificial Sequence
  mRNA sequence of the engineered mouse CD38 after humanization
gcaggctctgacccagtcagccctgtgctctcttcctgcctagcctgggccagtcttcgggagcccaatggctaactatgaatttagccaggtgtctggggacagacctggctgccgcctctctaggaaagcccagatcggtctcggagtgggtctcctggtcctgatcgccttggtagtagggatcgtggtcatacttctgaggccgcgctcactcctggtgtggactggagagcctaccacgaagcacttttctgacatcttcctgggacgctgcctcatctacactcagatcctccggccggagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgaaggatctggatcttagatcacctgtagcctggactgagatgaaggggctcagaagcaacactggtggaaagctgaaactgtcagggagaagcctctactacagtgttaacaccagagatggaagaacttcccaattctctgtgtactaccaacattcaagaaaaattactccataaaccagagttaaacttctatattgttatattagtctaactttctcatgtggtgcttctgtattgtttatatattgcttacatccttttattcctcttttaatgatctctcttttctctctctctctctctctctctctctctcaatgaggctgagaatccaacctgagaactttcatacatggtggataagcctatataccactgagctaaatcctcagcacagctgataacatcatttttgctgaaaaatggccaatcaaacttcccattagacaaagaaagtcaaatgtcaagtatatcgaaatgaatgacccttttttttttttatgttttttcattcttcccacagatattcacatggtaaacctgaggtcatagggtcattatagggaaggtgctgtgtgggaactacccacgtgccctgtgctttaatctttaactcaacacatccctgataactttgagcattcttttcttttcttttctttccttttcttttcttttcttttctttttcttttttttaaacagaagaaaatggagtttagagagataattgattttcctaaagtcacattcttaagatcatggttacatctagaattaaatcagttctgtcaaagtccacaacatatgatagtttttttcttttccgagcttttattgccaaattggtagtagcctgtgttgtcgtctagcaaatgctattgcattagattgaatataatagaatctgaaatgtgatctttatggaagatcacaaaactaattacttgcaccctctttctcttccacttcattcttggtgccttttataggcagtcaagagttaacatatatttgttgctaatattggcaatttatgttttctatcctttttttcttagatcagtcttgctcagaatcactggttttgttatgttgttgttgttgctattgttattgttcttgtctttttcaaagctcaaatctttaactttcctcatttatttatttaattgagctttagtctttatttttagaaacatagaaatttcttctagctatctggaatatcttcagttttttgaggcaaatgcttagaccattgatatttcagtctgttttctacacatgtactttaggattctaggtttctccctgagccctgctttcgatgtaacactgaatttctgtatgtctttactggttagttactttgatagtttgtatatgcttgacccagtgagtggcaggattagaaggtatggccttgctggaataggtgtgccactgtgggtgtagtcttaagacccttaccctagctgcctggaggccactattccactaacagccttcaaatgaaaatataaaactctcagctctgcctgtgccatgcctgcctggatgctgccatgctcccaccttgatgataatggactgaacctctgaacctgtaagccagccccaatttgttgtccttataaaagacttgctttggtcatggtatctgttcacagcagaaagaacctaactaagacagttaccattcagttcaaaataattcttgattttattgttatttagacatgtgatatttacttttcaacatctggagaattgtttaggttttttttgtgtgtgtgtctttagtaggtattaataaactaaattgagacaactccaaagtatttgcttggtttattgaaaatagcataaacagagccaggtgcttctgtattcatgttttgacaccagtttgtgattgttgaacaatatatcttatttctaaacttttaaaaagctacttaaagacattaacttgctcccttacaatatgttgatggttcagtccattaaatgcttactttgtaagt

SEQ ID NO: 13 - protein, 304 aa, Artificial Sequence
  encoded protein sequence of the engineered mouse CD38 after humanization
MANYEFSQVSGDRPGCRLSRKAQIGLGVGLLVLIALVVGIVVILLRPRSLLVWTGEPTTKHFSDIFLGRCLIYTQILRPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 14 - DNA, 27 bp, Artificial Sequence
  primer
attctctgcaaattaccaactcttcca

SEQ ID NO: 15 - DNA, 24 bp, Artificial Sequence
  primer
gttacatcgaaagcagggctcagg

SEQ ID NO: 16 - DNA, 26 bp, Artificial Sequence
  primer
atcagtcttgctcagaatcactggtt

SEQ ID NO: 17 - DNA, 25 bp, Artificial Sequence
  primer
ggttgttgggacagttttcactcca

SEQ ID NO: 18 - DNA, 1370 bp, Artificial Sequence
  5′ homologous arm
taagagaagcatagaaataatttgtgaaggacgtgagtttcagcacttaaataagagtttgctgatgagaaggtctgctgtgtggcagggtctcagggcaggataactgctctttcccaccacgcttccagaagtggaaaatgttctctccttcttccatcttcttctgtttcaagtcctgcagatgctccaaaggaaagacgtagagatttttatgacggttgggacttagaacagagagttcttgagaaggccactgcattaattaccagaaagaaaacataaaccctggaagccgggaagccgccaaacgtctattttctcaattgcctgtgatggtactctataaattttgattgacactaaagtacatgatgtcatcacttttcctttttatgtatgtaaacggtacggaaggagagggccctaatggtttcctggcatcgttttcaaaggcaggtgctgatgaacttaagcttctcagtctagactctggaaagaaaccccatcaaacctttcacattttcatttgtatcccctgattctcaacccgaactagggtcagaactgcctggggtgggggtgggggtggggtggaagtgggaggtatgaatgccactgatgatcccgcccaacattaccaagcccacctgcagtaaccctacctacttaaagttttagtctgacacagagtaattggacatatttatgagtacaatgtggcatttggcctatgtatacactgtataaagaacagtgacagacagctctatcgcctcaggtgctcatgacttctctgtgttgggaacattcggtcctctctacttgctgttttgaaacattgcccttactgggggtactaaagaaccttaaggactttttttatacaaggtgtagtatgagacagaaaacgctggtgtcttcctattttcttcttaatgttattagatgcttgggtcattttaatttcacagagcctcagcctcaggtaattttcatctcacagggacattttacacagctaataggatggtgtcaagaactgactgcccctgggtatatgattccagatgaagggtatttcttgttgctatctatgttccaaaaatattttttaaactttttcaatgtctttctttttttcttttatttattttttttatatgttcctaagattgatatttattttacatgtagagttgttttgtctgtagatatgttcatgcaccatgtatgtgcagtgactgtggaagccagatggaggtgttagctctggaaactgaacacactgttgggagccaccgtgagccatctctccagactcttcaaaaatatttttaaaatctgttttccatctttcattttataga

SEQ ID NO: 19 - DNA, 1395 bp, Artificial Sequence
  3′ homologous arm
taagacatccccaaatccagtctctccctcactttccctctctacatccttcctgttctccccttttcctgcctcttccactttcctcttttccctcctccctctccactttcttctttctcttctgcacttttggcaagaaatctcctccttaacctttatatatgtatttacacacacacacacacacacacacacacacacacacgttatatattcttgaacataactctaagtcatttttttaaaaaaatttaaattaaaattatatatatttttatctgtacagagtaatattggatataaatagacatagtgaacggattgttatctgtgagctatttaacatggacaactttgggtaccatcttaaggtgatctctttctttccccttcatagccagtgatgatctatttgcctagagagccatctgtgcatctcccctcacactgccttttagcaccatcacaccctctccatcctgctgggaatgctataaggttttactggtcactctttaccctcctaggtcagtctccatctgacaccatagtttttctcagagtgaaaatgggatgatgtcgatctccctctagaacctaacaatctctcttttgactctaggctaatggtctaaagacctgacctctgtgtttcagatgcctcaccttctcattcccttctccatcctcttttctgatcaaagctcatcatggctctttgcctttgcaccgtgccataggcacaaggtgcccttctctttagagtctgtctgaagacagtgattcttcttcaaagtaaatttcaacgtgaatctgtctctgattatttgacttcactacatcacatgtcctatgaggcagggctcccacctgattcaaccctctatctccagaacctaaccttcagtaaatgtttatatatatatgtgtgtgtgtgtgtgtgtgtgtttatatatatatgtatatatatatgtatatatatatatatatgcaaataaccttgagaacagtgtcatagaaaccatgcttctactctatactttgcaccacactaagggctaaacctgttttggtagggttgactatagaaagcctgagatcacctgagcccccaaatttatggaggttgaaaaggaaactctccataaaagagaattgccaggaatcagaggactatctgggggaaagtgtcttcttcccgaaaaggcctggccttctccctaagtgttcctggactccaggggagtgagaggggccggtgtctttagggtgcacttttgttgccatttcccagaatggtgagtcaaaacccatcacagtgttcctgtctgggatctggacggggaggtaaagagttggaaggaaatcaggggacacttacttaaattgggt

SEQ ID NO: 20 - DNA, 23 bp, Artificial Sequence
  sgRNA targeting site
tgagtgaccaatttaacaagtgg

SEQ ID NO: 21 - DNA, 23 bp, Artificial Sequence
  sgRNA targeting site
tgaatgtactcagtatctcctgg

SEQ ID NO: 22 - DNA, 23 bp, Artificial Sequence
  sgRNA targeting site
tgtgatgttgcaagggttcttgg

SEQ ID NO: 23 - DNA, 23 bp, Artificial Sequence
  sgRNA targeting site
gccctcattaccttgttacatgg

SEQ ID NO: 24 - DNA, 23 bp, Artificial Sequence
  sgRNA targeting site
gagtgaccaatttaacaagtggg

SEQ ID NO: 25 - DNA, 23 bp, Artificial Sequence
  sgRNA targeting site
tcaaaccataccatgtaacaagg

SEQ ID NO: 26 - DNA, 23 bp, Artificial Sequence
  sgRNA targeting site
gagatactgagtacattcaaagg

SEQ ID NO: 27 - DNA, 23 bp, Artificial Sequence
  sgRNA targeting site
tcttctcttgtgatgttgcaagg

SEQ ID NO: 28 - DNA, 23 bp, Artificial Sequence
  sgRNA targeting site
agggaatttacccccatgaatgg

SEQ ID NO: 29 - DNA, 23 bp, Artificial Sequence
  sgRNA targeting site
atgagctcaactccatttagagg

SEQ ID NO: 30 - DNA, 23 bp, Artificial Sequence
  sgRNA targeting site
gctactttataaggctgttgagg

SEQ ID NO: 31 - DNA, 23 bp, Artificial Sequence
  sgRNA targeting site
taaatggagttgagctcatgagg

SEQ ID NO: 32 - DNA, 23 bp, Artificial Sequence
  sgRNA targeting site
ctagattagtgatcacaaaaagg

SEQ ID NO: 33 - DNA, 23 bp, Artificial Sequence
  sgRNA targeting site
attcagcttaatgggaacattgg

SEQ ID NO: 34 - DNA, 23 bp, Artificial Sequence
  sgRNA targeting site
ggattaaaaatccattcatgggg

SEQ ID NO: 35 - DNA, 20 bp, Artificial Sequence
  sgRNA2 upstream sequence
tgaatgtactcagtatctcc

SEQ ID NO: 36 - DNA, 24 bp, Artificial Sequence
  sgRNA2 forward oligonucleotide
taggtgaatgtactcagtatctcc

SEQ ID NO: 37 - DNA, 20 bp, Artificial Sequence
  sgRNA2 downstream sequence
ggagatactgagtacattca

SEQ ID NO: 38 - DNA, 24 bp, Artificial Sequence
  sgRNA2 reverse oligonucleotide
aaacggagatactgagtacattca

SEQ ID NO: 39 - DNA, 20 bp, Artificial Sequence
  sgRNA11 upstream sequence
gctactttataaggctgttg

SEQ ID NO: 40 - DNA, 23 bp, Artificial Sequence
  sgRNA11 forward oligonucleotide
taggctactttataaggctgttg

SEQ ID NO: 41 - DNA, 19 bp, Artificial Sequence
  sgRNA11 downstream sequence
caacagccttataaagtag

SEQ ID NO: 42 - DNA, 23 bp, Artificial Sequence
  sgRNA11 reverse oligonucleotide
aaaccaacagccttataaagtag

SEQ ID NO: 43 - DNA, 132 bp, Artificial Sequence
  DNA fragment containing the T7 promoter and sgRNA scaffold
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 44 - DNA, 25 bp, Artificial Sequence
  primer
ctaggcacttagcaggatgcccttg

SEQ ID NO: 45 - DNA, 25 bp, Artificial Sequence
  primer
cctgaagcccaaggatgtgaaagga

SEQ ID NO: 46 - DNA, 23 bp, Artificial Sequence
  primer
aactgatgaatgggagcagtggt

SEQ ID NO: 47 - DNA, 23 bp, Artificial Sequence
  primer
gcagacactctatgcctgtgtgg

SEQ ID NO: 48 - DNA, 25 bp, Artificial Sequence
  primer
accatgtatgtgcagtgactgtgga

SEQ ID NO: 49 - DNA, 24 bp, Artificial Sequence
  primer
cacagataacaatccgttcactat

SEQ ID NO: 50 - DNA, 76 bp, Artificial Sequence
  connection between the downstream of the mouse 3′UTR and the upstream of the LoxP STOP sequence
gatggttcagtccattaaatgcttactttgtaagtgtcgacattaagggttccggatcctcggggacaccaaatat

SEQ ID NO: 51 - DNA, 1594 bp, Artificial Sequence
  DNA sequence of the humanized mouse CD38 gene (chimeric CD38 gene DNA)
gtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 52 - DNA, 4730 bp, Artificial Sequence
  5′ homologous arm
ctgctgtgacggttaatatcgactctggaatcatctaggagacatgtcattggcatgttttcggaagatttttctagattgagctagttgaagtagaaagactaatcataagtaaggataatatggataaatatcccaaccattctatcatttgggaaccctagtctaaataaaaagaatgtgaactgaacgatagttttcattgctctaagcttcctgaccaaagaccagctctctcatgctccctccatatcgcctctgccacggtggacaagactaatgttgagtttgacctgtgaagtcacggaaggataaatgtcctgggctcttataatcctgcagtcccattagaatcatggcagcaataacttgtgtcttttcactgttttttatctttcttttgtgctggaggatttaggagccatggtctattatttaaatctatccttgctcaaatttcagtttggtgtttacccagtgttaaggataacaggacttcatgacaactcccataattcttaggtctaagccctaaacttcatgtctcaaaaggcagctgtattagaagatagcccagttacctgtatagaataattaaactgagaccatggagggagggaaggctcaaatccatcacctggtggtttcatacaaactggaaattagatcgcagactgttacagacatacctgctcagcgaataaagacgaagagaagggcagactgaaagccgagtggacaggcctcagaacgagccacccatgcagaaaccttgatcctgaacatccagtagtgcaagacaatacatttcttttgcttaagacacccagtgtgtgtgatactgtgctatggcatctccaacagactggctgactgagcttagacctttgtccattttcttttgttgttgttgttttgttttgttttttgtttttgtctaggcagtgggtgtggttgcacaaacgggggtggggttgggggtggggcgggagggggcggggttcactaactggcctttctttgaacttttaaccatttaatgacaccctgaagacttgtctactccccagcctgactagtagtctccatcagtgttccaaacagttcctcacaggagtgttctctttctacttcatgaaagaagcaatcaggagacaacttccttgtcaaaacccccacccttgcatattctccacctctttttgtaaggagctggagcttcagcccacatccccaccgtgcctcagcttcctctccccatctcctgcatttgtcaagtgttactgtgtgagtcatcttgtcagcctccaaaagtcctgtgagaagtctggtctcagaacaggatgctgtttcctgactcccaggctattgttactttctcattgttgaggaaaaaatatccaacgtgacaactgaaaagaaggaaatttcactttggttcaggttttagaggatttggtccatggtctcttggcttcatggtttttttgtttgtttgtttgttttggttttttttggtttttgtttttggtttttcaagacagggtttctctgtgtagccctgactgtcctagaactcactttgtagaccaggctggcctcgaactcagaaatccgcctgcctctgcctcccgagtgctgggattaaaggcatgcgccaccacgcccggcaacttcatgcttttggtagagcattatatggcatagaggaatagttcacatcatgaccaaccagaaggcagagaactggacaggaagttgtcagggatagcacagtacagcatcaaagacttttaacgagtaacccaagtcctccatcttggcccatcctcctaaattcataaaccttctaaaatagttccccaacctgaggtccaagagttctaaaatcatgatcctatagagaattttacatatccacatcataatgcccagcatcttccaattgcatccttatttctctgttccttttgacactaagcaagcaaacaaacaaacaaacaaaatggctccctttttcaacacaactgcaccctaacttactaagagaaccttcaaaccttctcttgaacctacctgctggactgtgtccttctcattccctgaaaatctgaaaatgttccctctcgaggacggaaagaagcatttaagactttgctgttcttgcagaaaactggacttaggccctagcatccgcatcaggcagcttacagccatttacgacttcaattctgagggacctgatgccctactttggcccatatggccatcccacataagagcctacacatacatgcacaaataatatgttgttttttaaaattaattaattaattttttgttagttaagaacttctttctttttcatgagtcacccttccaagtttcctttgctgggattttcacacttttctgactcttacgtcaagaatgttccccaaggttttaaaacttctccattccatctgttacatagtctttctgtgacatgacgaccctctttacaaactgtgctgaaacttcatgtgccaggtcatttaaggtcactgatcttaaacaggttaaaacagaattctgtgctgataaaatggcctgacctgttcagtcttctctgctgtcagacctgatgaccccagtttaatgccttggacacacatggcagagagagagaaaggacccctcaaaggtgtccttggtcctccattcatgtcctgtggcatatattcacacacacacacacacacacacacacacacacacacacacacacagagagagagagagagagagagagagagagagagagagagagagagagagaaccaaagaactgcaggaacaaaaatgaaggtccagttaacaggcccagctcaaggggaggctccaaggcctgacattattactgatgctatggagtgcttacaaacaagggcctaacatgactgcctttcgaaaggcccaacaagcagctgaatgagtccgatgcagatatttacacccaatcaatgcacagaagctggtgacccctgtggttaaattagggaaaagctagaagaagctgaggaggagggcaaccctataagaacagcagtctcaactaacctggaccttcgagaccactcagacactgagccaccaaccaggcagcacacaccagctgatatgaggtccctaacacatatacagcagaagagtgctgggtctggactcagtcagagaagatgcacctaaccctctagatacttggggccccagggagtgaggaagtctggtggggtggggggtagggataggggtggggacatcctcttggagagggggtggggaggaggaggtgagggaatgtaccaggagggagatgaagtctggactgtaaaaaaagattaaagaatgtatttttaaaaaagaaaaagaaaacacaggaaaaaaaagaaagagagggagagaggcacacaaaatgaataagcaaaatgacagcaaacgtttaaaaaaaaatgaaatgcacagtctaaaaaagtcctcctgacatacggagctgcagagacttactctcctgctgccttcggaaggattctctgtcatcctgtagtatagctgcaatgctgctttcccgcatcttaaaacagaacaaggggctttcctgccccagctcttgcatccagtaccctctgcctgaaaagtgtcccgtgaaaatcctgtgtcatgatccattctgtgacaagtctctgtttaagcagaagagtgcggggtcttcttgggaaggagagccaggaagagcctttgtatttggggttcccatgtgggatgaagtattactgcacatgagatgggttgcgagcatcctatcaactctaaaatcctacctttgtcaaccccctagagtaagcagcaataacttacatttattgagtaatcggttgttatgtcttagggattatgctaaacacctaggaggtattaagccatctaatcaaagcaagctttgatgtaatgaatgtgaggctctgctcacatctatctagttcagagaggtaagtttatcttaacagcactttaccatctggggctgggtttgaactgatacctcctgagaagaggggcagagtaaagctgagactttaaagatacttgggcaggatttttatcaaagctcagaagtagaggtaagcaggccattcattgccaggcatttgggtgaacagagagagagagggagggaggagagggagagggagaataattttacacacacacatacacacacacctgagcataagtagcaaacatgtcaagtgtcaaggttgttggtcaggagtattctgaagctgttgtttgtgtaccagcagaggctaaagtgctgtcttctcttggagagagaggttctggtcagtttgctctggagtctggaagtaagcagttggagtgcctgttgacaacccaagagccttattagtccagtcacctccgtggaactttggagcaggagtagggggtgggaaaggaagcagataaaagcaagtgaaaaaaaggaggaggggcaggctctgacccagtcagccctgtgctctcttcctgcctagcctgggccagtcttcgggagcccaatggctaactatgaatttagccaggtgtctggggacagacctggctgccgcctctctaggaaagcccagatcggtctcggagtgggtctcctggtcctgatcgccttggtagtagggatcgtggtc

SEQ ID NO: 53 - DNA, 3783 bp, Artificial Sequence
  3′ homologous arm
aggtgagttggcttctgaggctcactctaggcacagtgcgcagcgggacaccggggaagcaggctgtgactcagctggagctctcgcggcgggggggaatcttccagattgtcccaaacaaagaatcttgtgttgtggatcagctggaagccgaagggcagtgctgagcggaaggtctcctgttcaagttaccagaacaccgcctgcacaatgcctcctccgttttggcagtgtctgtgatgggggcgccgatccttgctgagctctgcaaagatgctcgcttcccacagagggtcctgaactctgaaggaacccttagggctatctttggccacagccatgcttctgggacaaagattactctcaggcacaggaacttcacattgtcttttggcttgtggtttccatcagacaccgcccctgcccagaaagttaaggtcacaggcgacatttaactaggtggcttgctcagcttgctgtgggggtggaaggggccaggaggatttttgtgaattcaaggttaacaagggctgcacagcaacattgtaaattccaggacaagcttgggttacaaagagagacctgactcaaaataagttaattaattttaattaattacaacacaaagaaaacaaaaaacaagcccaaacagcatcctggtctttttttttttttgcacctgatcattaataaaaacaaaacaagacagactgtcaaaactgaaatattactcatcaggtaagcgaaatccacttggtgatcctgtgtagacccttgggaacatcatcttcattggtcaatagagtcagcccatagaaccccttccacccactacctgccccagagggaatgatttatgttgcttctccctggcatcattttaacgtagaactgatttctgaagtcaaacagaaagcccagcaggctgggaaggcacacgcagaaacccaacttcacattctgtcttcagatgcacacacttcccgggtgctcgaatgctcagtggtttgggtcctggtcttacttgaaaatgttggagtataaacactctcagggtcctgataaatattttgtttacagatgtttaaatgatattggttgttcagccattttgatagcttgtaaattttctgtgcttagcaaaccagatgcatggcactggcatgatgtgtgcttagcattaaccatgtggtgtagacggtaagcacttcttcaacctatccactcatgtgtatatgtgtgcattcagcaagaattaattatttgtgtttatcaaataataatcaatctaaaaataacaggattaactaataaaagtatatatatttactctgtgtgtgtacatgtgtgtgtgtgtgtgcacacacgctcgtaagtgcgtgagtgcctgttccacagtacacgtgcagaaatcatggaattcagttctttgcttccactctgtagcttctgggaatcaaattcagataattagaggtttggaagcaagcatccctacctgctagtctgtatcatttgcctaacatattactattgtgatgagcatcttttcagacatctcacacacacacacacacacacacacacacacacacacacacacacacactttatatgaatagattagtggcaggtacatctcatgtgtcaggcacttatgtcaggagctgaggtgcagctcagtgatagagctctggcctggtttacaaagtgccctgggttcaccctcagcatcgcagtagaggaatgagtggaacagaatcaaccaggacagaaggcaacgatacatagacggttgctattgatatctccattttatagataagaaaagggagtcaaaagctgaaccctgttgagccctgaagccagatcattggagctggaatcttggctctgttgcgacctcagaccccgccctcagtgtatctgcgctgtggtgtgttcccaccttgtaaaatgggaacgttaatggcagcactcggcatctagagtaaacgaagaggactaagatgaataatcttgtcatggtaaatgctcaaaatgttactgctgtgtaaatggagtgttgcagatacctctccacaatagcaaatataaaataagtacaggcgttcatgcattagaacattgttgtataattcagtcctccagtgctgaaagttacatcgtttcgaatcttgttcacctaacacttggtggtgatagggtctctcatattgctacacccaaatctaccttattttttatagcgtagatgtaaccatttccagtcatcaaaaatttaatttccaattttaaattacatatctctgttacatttaggggatgggaggattgccatagggcatgcatggaagttggttctttcttcccctatgtaggttctgggaatcaaactcaggtctttaggcttggcagcaatcacctttatttaaatgagacatcgtgctggcccattttctaatttttcaatcccattcattcattcattagatatttttgagcctctgctgggttccaggtgggtgatctggctactggtggcacaggaggggtagtgtcctgattcttgtccagctttcccttacagtaaacatcttcaacttataccttcatgtgtgcactggagcgttctgtatgacagactcccactgatagaatgagattgtacatgttttagttagggtttctattgctatgaagagataccacgagcacagcatatcttgtaaatgaaaatatttaattggggctgacttacagttcagaagttttagttcattatcatgatgatggcatacaggcagacatagtgatggagaaggagctgagagttctacatctggactgacatacaacaggaagacagacagacacttggcctgccttgagcatctaaaaaccacaaagcccactcacacacttcctccaataaggccacgcctactccaacaagaccacacctcctaatagtaccactctctatggacctttgggggccattttcactcaggtcaccacagtgcatattttgtttgagtggctttcaaattgcacatgtttctaaatcctctgtgaaatagttttattagttatttattaactctcatatcagtctacagggcccacttctccatgatatgaccattattaaatgtcttaaatctttttaaattatgagaaagatggaaagataacatataaccattccctgtttgccagcaagtcttctgtctggcttttgtccatttaaactttgttcagatcttcactctttctgttgagttaattacatatgtgattttgggttgttcatattctagataatatttatacacacagacacaaagtatctcacagctacatcactaaggcctccaccccaaggacacattttactgacaacaccctgagtttcattattatattggggctacttcttattttgatacccttactaagaagcaccgagaaagccagttttattttcaaacctagtgcactggaaatgtttactctctgttctgcactatgaatcaggaattctttcctctctgggtctgcactttctcttgcacagctagaaagcaaagaagcgaccctcagcttctgcactaagagtttgagccacagctttgggttcctagggggagttatgcatcttccctttggcagttgacaagagttacatcttacatcacttctgcaaggggctgtgtttcttccagcctccaatgaccacttcctgtgtgtccatcaggttagcatgaacagtccctcctt

SEQ ID NO: 54 - DNA, 777 bp, Artificial Sequence
  the nucleotide sequence 214-990 of NCBI accession number NM_001775.4
gtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctga

SEQ ID NO: 55 - DNA, 80 bp, Artificial Sequence
  connection between the upstream of the human CD38 gene sequence and the mouse CD38 gene locus
ctcctggtcctgatcgccttggtagtagggatcgtggtcgtcccgaggtggcgccagcagtggagcggtccgggcaccac

SEQ ID NO: 56 - DNA, 80 bp, Artificial Sequence
  connection between the downstream of the Neo cassette and the mouse CD38 gene locus
gaacttcatcagtcaggtacataatggtggatccccatggaggtgagttggcttctgaggctcactctaggcacagtgcg

SEQ ID NO: 57 - DNA, 1787 bp, Artificial Sequence
  mRNA sequence of the engineered mouse CD38 after humanization
gcaggctctgacccagtcagccctgtgctctcttcctgcctagcctgggccagtcttcgggagcccaatggctaactatgaatttagccaggtgtctggggacagacctggctgccgcctctctaggaaagcccagatcggtctcggagtgggtctcctggtcctgatcgccttggtagtagggatcgtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 58 - protein, 300 aa, Artificial Sequence
  encoded protein sequence of the engineered mouse CD38 after humanization
MANYEFSQVSGDRPGCRLSRKAQIGLGVGLLVLIALVVGIVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 59 - protein, 303 aa, Rattus norvegicus
MANYEFSQVSEDRPGCRLTRKAQIGLGVGLLLLVALVVVVVIVLWPRSPLVWKGKPTTKHFADIILGRCLIYTQILRPEMRDQDCKKILSTFKRGFISKNPCNITNEDYAPLVKLVTQTIPCNKTLFWSKSKHLAHQYTWIQGKMFTLEDTLLGYIADDLRWCGDPSTSDMNYDSCPHWSENCPNNPVAVFWNVISQKFAEDACGVVQVMLNGSLSEPFYRNSTFGSVEVFNLDPNKVHKLQAWVMHDIKGTSSNACSSPSINELKSIVNKRNMIFACQDNYRPVRFLQCVKNPEHPSCRLNV